(19) United States
(12) Patent Application Publication
(10) Pub. No.: US 2007/0071773 A1
(43) Pub. Date: Mar. 29, 2007

(54) Compositions and methods for the treatment and prophylaxis of infections caused by gram positive bacteria

(76) Inventors: Emanuel Hanski, Zur Hadassah (IL); Allon E. Moses, Jerusalem (IL); Carlos Hidalgo-Grass, Modiin (IL)

Correspondence Address: COOPER & DUNHAM, LLP, 1185 AVENUE OF THE AMERICAS, NEW YORK, NY, 10036, US

(21) Appl. No.: 10/525,178

(22) PCT Filed: Aug. 19, 2003
(86) PCT No.: PCT/IL03/00687

§ 371 Date: Mar. 31, 2006

(30) Foreign Application Priority Data
Aug. 22, 2002 (IL) 151436

(51) Int. Cl. A61K 39/09; C07K 14/315

(52) U.S. Cl. 424/244.1; 530/330

(57) ABSTRACT

The invention relates to methods of treatment, compositions and uses of an isolated and purified peptide for the inhibition of an invasive and/or non-invasive infection of Gram-positive pathogenic bacteria. A specific peptide comprises an amino acid sequence substantially homologous to the amino acid sequence encoded by the SilCR ORF of the sil locus at position 2985 to 2863 of the complimentary strand of the genomic sequence of Group A Streptococcus (GAS) bacteria specified in GenBank accession number AF493605. Preferably, the peptide of the invention comprises an amino acid sequence DIFKLVIDHISMKARKK, also denoted by SEQ ID NO: 32 or any functional fragment, analog or derivative thereof. The invention further relates to methods for inhibiting an invasive and/or non-invasive infection of Gram-positive pathogenic bacteria, using an agent such as the peptide of the invention, which induces down regulation and/or inhibition of chemokine protease activity.

### FIELD OF THE INVENTION

The present invention relates to prevention and treatment of infections caused by Gram-positive pathogenic bacteria. More particularly, the invention relates to an isolated and purified peptide capable of inhibiting an invasive and/or non-invasive infection of pathogenic bacteria, preferably a Group A Streptococcus (GAS) bacteria and to compositions and methods thereof.

### BACKGROUND OF THE INVENTION

Group A streptococcus (GAS) is a Gram-positive pathogen globally responsible for human morbidity and mortality. GAS causes extremely variable clinical manifestations ranging from mild self-limiting superficial infections of the skin and mucosa, to life-threatening invasive infections such as septicemia, toxic shock syndrome and necrotizing fasciitis (NF). The organism is also responsible for acute rheumatic fever and acute glomerulonephritis [Bisno, A. L. et al. Engl. J. Med. 334:240-245 (1996); Musser, J. M. et al. In R. M. Krause and A. Fauci (ed.), Emerging infections. Academic Press, San Diego, Calif. Pp. 185-218 (1998)]. A marked increase in the incidence of TSS and NF in the last two decades has been reported in several countries including the USA, Canada, Northern Europe, New Zealand, Australia and Israel [O'Brien, K. L. et al. Clin. Infect. Dis. 35(3):268-76 (2002); Moses, A. E. et al. Emerg. Infect. Dis. 8(4):421-6 (2002)]. Despite prompt antibiotic treatments and surgical debridement, GAS TSS and NF are associated with high mortality rates ranging from 20% to 60% [Davies, H. D. et al. N. Engl. J. Med. 335(8):547-54 (1996)]. The reemergence of invasive GAS infections has renewed interest in the basic mechanisms of GAS pathogenesis and spurred efforts to identify and develop new treatment approaches [Bisno, A. L. et al. Lancet Infect. Dis. 3(4):191-200 (2003)].

GAS infection is defined as invasive when bacteria are isolated from blood or other sterile tissues. A prospective population-based study (PPBS) in Ontario, Canada showed that the annual incidence of GAS invasive infections is 1.5 cases per 100,000 persons [Davies, H. D. et al. N. Engl. J. Med. 335:547-54 (1996)]. Recently, a PPBS study was conducted in Israel, showing that the overall annual incidence of invasive GAS infections is even higher, reaching 3.7 cases per 100,000 persons [Moses, A. E. et al. Emerg. Infect. Dis. 8:421-426 (2002)].

Worldwide, epidemiological studies revealed that most outbreaks of invasive GAS diseases are caused by strains within the M1 and M3 serotypes [Davies (1996) ibid.]. In a recent prospective, population-based study of invasive GAS infections in Israel, the present inventors have found that a high proportion of NF causing isolates belonged to the M14 serotype [Moses, J. et al. Clin. Microbiol. In press (2003)]. The emergence of exceptionally widespread and virulent GAS clones suggested that the increased invasiveness might be associated with specific genetic elements that have been acquired by horizontal transmission [Cleary, P. P. et al. Lancet 389(8792):518-21 (1992); Musser, J. M. et al. Dev. Biol. Stand. 85:209-13 (1995)] or phage acquisition [Banks, D. J. et al. Trends Microbiol. 10(11):515-21 (2002)]. Conversely, the fact that genetically indistinguishable clones can be isolated from invasive infections as well as non-invasive infections indicates that host factors might play a role in determining disease outcome [Chatellier, S. et al. Infect. Immun. 68(6):3523-34 (2000)]. A recent report suggested that allelic variation of human leukocytes antigen II contributes to the differences in severity of GAS infections [Kotb, M. et al. Nat. Med. 8(12):1398-404 (2002)].

Other studies demonstrated that additional serotypes caused invasive infections [Norgren, M. et al. J. Infect. Dis. 166:1014-20 (1992); Stanley, J. et al. J. Clin. Microbiol. 33:2860-5 (1995); Chaussee, M. S. et al. J. Infect. Dis. 173:901-8 (1996); Muotiala, A. et al. J. Infect. Dis. 75:392-9 (1997)]. Furthermore, the clonal M1T1 strains could be isolated from patients with severe and non-severe invasive infections [Chatellier, S. et al. Infect. Immun. 68:3623-34 (2000)]. These reports emphasize the role of host response in determining the severity of GAS invasive diseases.

The hallmark of severe invasive GAS infections such as NF is an aggressive and rapid spread of the bacteria. Ashbaugh, et al. [J. Clin. Invest. 102:550-60 (1998)], have developed a murine model resembling human NF. Mice inoculated subcutaneously with a NF-causing strain, developed a spreading tissue necrosis, became bacteremic, and subsequently died. In this model M protein and hyaluronic acid capsule (HAC) are absolutely required for mouse mortality. Furthermore, HAC production is induced upon introduction of GAS into the pharynx of baboons or into peritoneum of mice [Gryllos, I. et al. Mol. Microbiol. 42:61-74 (2001)]. Also, it was demonstrated that cysteine protease (SpeB), streptolysin S (SLS), and C5a peptidase also contribute to tissue necrosis and bacterial spreading [Lukomski, S. et al. J. Clin Invest. 99:2574-2580 (1997); Lukomski, S. et al. Infect. Immun. 67:1779-88 (1999); Kuo, C. F. et al. Infect. Immun. 66:3931-5 (1998)].

Pathogenic bacteria modulate virulence gene expression in response to the rapidly changing environments encountered during the course of infections [Chiang, L. et al. Annu. Rev. Microbiol. 53:129-54 (1999)]. GAS can survive and multiply in diverse anatomic sites, particularly during aggressive invasive diseases. To identify genes contributing to GAS ability to spread, the inventors developed the polymorphic-tag-lengths-transposon-mutagenesis (PTTM) method. This method resembles the original signature-tagged mutagenesis (STM) method, which has been successfully used both in Gram-negative and Gram-positive pathogens to identify genes that contribute to virulence in-vivo [Mecsas, J. Curr. Opin. Microbiol. 5:33-37 (2002)]. Using the murine model, a mutant of a strain causing NF (emm14 type (M14)) that was severely attenuated in the ability to spread from the skin to the spleen and to cause a lethal infection has been identified. The mutant contained a transposon insertion in a locus that was termed sil, for streptococcal invasion locus. sil exhibits a high homology to the quorum-sensing competence regulon com and to the peptide-sensing system blp of Streptococcus pneumoniae [Hui, F. M. et al. Gene 163:25-31 (1995); De Saizieu, A. et al. J. Bacteriol. 182:4696-703 (2000)]. Moreover, the inventors further demonstrate that sil is also involved in DNA transfer.

Recently, the genome of the M18 strain MGAS8232 was sequenced and a highly homologues region to sil locus was identified [Smoot, J. C. et al. Proc. Natl. Acad Sci. U.S.A 99(7):4668-4673 (2002)]. In this strain, SpyM18_{—}0540 (designated by the inventors silCR), which partially overlaps silC (FIG. 3B), was suggested to be transcribed from the reverse strand. SilCR encodes a predicted 41 amino acid peptide with a leader containing a double-glycine and the motif RKK at the C-terminal [Smoot (2002) ibid.]. SilCR has characteristics of a bacterial pheromone peptide, including a conserved cleavage site and therefore the 41 amino acid peptide is processed to form a predicted 17 amino acid peptide [Havarstein, L. S. et al. Mol. Microbiol. 16(2):229-40 (1995)]. However, in M14 GAS a missense mutation changes the ATG start codon of the silCR gene suggesting that the peptide may not be produced [Hidalgo-Grass, C. et al. Mol. Microbiol. 46(1):87-99 (2002)]. silCR possessing an ATG start codon is present in the genome of M18 GAS [Smoot (2002) ibid.], which is rarely associated with invasive disease, while SilCR is completely absent in the M1 and M3 GAS genomes, which are commonly associated with invasive disease.

The inventors have surprisingly showed that in the JS95 virulent strain used in the present invention, the ATG initiation codon is replaced by ATA. Elimination of this initiation codon in the virulent strain prevents production of the 41 amino acid precursor peptide, and therefore the formation of the predicted 17 amino acid mature peptide. Since the sil locus was found in the present invention to be involved in invasiveness, the inventors next analyzed the potential effect of the peptide encoded by silCR in virulence. The inventors have surprisingly found that the 17 amino acid peptide exhibits protective properties against GAS infections.

The present application further includes an analysis performed in two patients with NF caused by M14 serotype GAS strains. As described in other clinical reports of NF [Barker, F. G. et al. J. Clin. Pathol. 40(3):335-41 (1987)], histopathologic examination of debrided tissue revealed a paucity of neutrophil infiltration. A similar histopathologic appearance was observed when these strains were tested in a murine model of NF. These strains released to the culture medium a protease that degraded chemokines responsible for neutrophil recruitment in humans and mice. The SilCR pheromone peptide induced down-regulation of the GAS chemokine protease activity. Neutrophil migration to tissues was enhanced, controlling GAS tissue invasiveness and protecting mice against this lethal infection.

Since an early use of antibiotics does not influence NF severity and mortality rate from such disease, which ranges between 20 to 70%, the peptide of the invention may be used for the treatment and/or prevention of GAS infections.

It is therefore an object of the invention to provide a protective peptide and to develop methods and compositions for inhibiting an invasive and/or non-invasive infection of Gram-positive bacteria and method and compositions for the treatment of pathologic disorders related to such infections, using the peptide of the invention. This peptide comprises an amino acid sequence substantially homologous to the amino acid sequence encoded by the SilCR ORF (open reading frame) of the sil locus at position 2986 to 2863 of the complementary strand of the genomic sequence of Group A Streptococcus (GAS) bacteria specified in GenBank accession number AF493605. This position defines the SilCR ORF, which encodes the precursor 41 amino acid peptide, which processed to form the mature 17 amino acid peptide of the invention (located at positions 2913 to 2863). Preferably, the peptide of the invention comprises the 17 amino acid sequence substantially as denoted by SEQ ID NO: 32 or any functional fragments, analogs and derivatives thereof. The invention further provides methods using this peptide and compositions thereof, for the treatment of GAS infections.

It is a further object of the invention to provide additional active peptides useful in the treatment of GAS infections.

Another object of the invention is to provide methods and compositions for inhibiting an invasive and/or non-invasive infections of Gram-positive bacteria and for the treatment of pathologic disorders relates to such infections, using an agent which induces down regulation of chemokine protease activity. As shown by the Examples, such agent may be for example the peptide of the invention or a serine protease inhibitor.

These and other objects of the invention will become apparent as the description proceeds.

### SUMMARY OF THE INVENTION

In a first aspect, the invention relates to an isolated and purified peptide capable of inhibiting an invasive and/or non-invasive infection of Gram-positive pathogenic bacteria comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605.

In one particular embodiment, the invention relates to an isolated and purified protective peptide capable of inhibiting an invasive infection of Gram-positive pathogenic bacteria. The peptide of the invention comprises an amino acid sequence substantially as denoted by SEQ ID NO: 32 or any functional fragment, analog or derivative thereof.

In a specifically preferred embodiment such bacteria may be Streptococcus sp. selected from the group consisting of Group A Streptococcus (GAS) bacteria and Group B Streptococcus bacteria.

In yet another embodiment, the isolated peptide of the invention is capable of inhibiting the spreading of a GAS bacteria and/or tissue necrosis and lethal effect caused by this bacteria.

According to a preferred embodiment, the peptide of the invention is capable of inhibiting invasive infection that commonly may lead to any one of soft tissue infection, bacteremia, septicemia, toxic shock syndrome (TSS) and necrotizing fasciitis (NE), and to non-invasive infection that may lead to rheumatic fever and/or acute glomerulonephritis.

In another specifically preferred embodiment, the peptide of the invention is capable of inhibiting an invasive and non-invasive infection caused by Gram positive bacteria that may be any one of GAS bacteria such as Streptococcus pyogenes, Staphylococcus aureus, Streptococcus pneumoniae and oral Streptococci. Preferably, the GAS bacteria may be any virulent strain of Streptococcus pyogenes.

The peptide of the invention or any fragment, analog or derivatives thereof may be in the form of a dimer, a multimer or in a constrained conformation. The peptide of the invention may be conformationally constrained by internal bridges, short range cyclization, extension or other chemical modification.

In a second aspect, the invention relates to a method of inhibiting an invasive infection of Gram-positive pathogenic bacteria in a mammalian subject, comprising administering to said patient an inhibitory effective amount of an isolated and purified peptide of the invention, which comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605, or any functional fragment, analog or derivative thereof or of a composition comprising the same.

According to one embodiment of this aspect, the Gram-positive pathogenic bacteria may be any Streptococcus sp., such as Group A Streptococcus (GAS) bacteria, Group B Streptococcus bacteria or Group G Streptococcus bacteria.

In yet another embodiment, the peptide used by the method of the invention is capable of inhibiting the spreading of GAS bacteria and/or tissue necrosis and lethal effect caused by said bacteria.

Still further, the invention relates to a method of preventing and/or treating a GAS invasive infection related pathologic disorder. According to one embodiment, this method of treatment comprises administering to a mammalian subject in need of such treatment a therapeutically effective amount of an isolated and purified peptide of the invention, any functional fragment, analog or derivative thereof or of a composition comprising the same. The peptide of the invention comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF498605.

In a specifically preferred embodiment, the peptide used by the methods of the invention comprises an amino acid sequence substantially as denoted by SEQ ID NO: 32 or any functional fragments, analogs or derivatives thereof.

In a specifically preferred embodiment, the method of the invention is intended for the treatment and/or prevention of a GAS invasive and non-invasive infection related pathologic disorder. Such disorder may be as a non-limiting example, a soft tissue infection, bacteremia, septicemia, toxic shock syndrome (TSS), necrotizing fasciitis (NF), rheumatic fever or acute glomerulonephritis.

According to a particular embodiment of the method of the invention, the effective amount of the isolated and purified peptide or of a composition comprising the same may be administered to a subject in need of such treatment, prior to potential exposure to said pathogenic bacteria.

More specifically, the effective amount of said isolated and purified peptide or composition comprising the same may be administered to a subject in need of such treatment, in a single dose or alternatively in multiple doses.

The effective amount of the isolated and purified peptide or of a composition comprising the same may be administered by the method of the invention to a subject in need of such treatment, by a single route of administration or alternatively by at least two different routes of administration.

The effective amount of the isolated and purified peptide of the invention or of composition comprising the same may be administered to a subject in need, by a route selected from oral, intravenous, parenteral, transdermal, subcutaneous, intravaginal, intranasal, mucosal, sublingual, topical and rectal administration and any combinations thereof. More preferably, the effective amount of said isolated and purified peptide or composition comprising the same may be administered to said subject subcutaneously.

According to a specifically preferred embodiment an effective amount of the peptide of the invention may range between 0.5 μg/kg to 100 mg/kg of body weight. Preferably, between 10 μg/kg to 10 mg/kg of body weight, most preferably, between 300 μg/kg to 5 mg/kg of body weight.

According to a specifically preferred embodiment, the method of the invention is intended for the treatment of a human patient.

Still further, the invention relates to a method for disinfecting an environment and/or preventing infection caused by Gram positive bacteria, comprising the step of applying a sufficient amount of a peptide of the invention or a composition comprising the same, onto a surface of a medical equipment, medical devices and disposables. The peptide of the invention used by this method comprises an amino acid sequence substantially homologous to the amino acid sequence encoded by the SilC ORF of the sil locus at position 2985 to 2863 of the complementary strand of the genomic sequence of Group A Streptococcus (GAS) bacteria specified in GenBank accession number AF493605 or any functional fragment, analog or derivative thereof.

In a further aspect the invention relates to the use of an isolated and purified peptide comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605 or any functional fragment, analog or derivative thereof, as an agent for inhibiting the invasive and non-invasive infection of a Gram-positive pathogenic bacteria. Preferably, such peptide comprises an amino acid sequence substantially as denoted by SEQ ID NO: 32 or any functional fragment, analog or derivative thereof.

The invention further provides the use of the peptide of the invention, in the preparation of a composition, which inhibits the invasive infection of Gram-positive pathogenic bacteria. Such composition comprises as an active ingredient an isolated and purified peptide of the invention or any functional fragment, analog or derivative thereof, in an amount effective to inhibit spreading, tissue necrosis and/or lethal effect caused by said bacteria.

Still further, the invention relates to the use of a peptide as defined by the invention, in the preparation of a pharmaceutical composition for the treatment and/or prevention of a GAS invasive infection related pathologic disorder.

According to a specific embodiment, the invention relates to the use of such peptide in the preparation of a composition for the treatment of a GAS invasive infection related pathologic disorder such as, soft tissue infection, bacteremia, septicemia, toxic shock syndrome (TSS) and necrotizing fasciitis (NF), and the non-invasive sequela such as rheumatic fever and acute glomerulonephritis.

The invention further relates to the peptide of the invention for use in the inhibition of invasive infection of Gram-positive pathogenic bacteria.

Still further, the invention relates to the peptide of the invention for use in the treatment and/or prevention of a GAS invasive infection related pathologic disorder. Such disorders may be for example, soft tissue infection, bacteremia, septicemia, toxic shock syndrome (TSS), necrotizing fasciitis (NF), rheumatic fever and acute glomerulonephritis.

The invention further provides for the use of the peptide defined by the invention for disinfecting an environment and/or preventing infection caused by Gram positive bacteria by applying a sufficient amount of a peptide of the invention or of a composition comprising the same, onto a surface of a medical equipment, medical devices and disposables.

In yet another aspect, the present invention relates to a composition which inhibits the invasive and non-invasive infection of Gram-positive pathogenic bacteria. The inhibitory composition of the invention comprises as an active ingredient an isolated and purified peptide as described by the invention, in an effective amount to inhibit spreading, tissue necrosis and/or lethal effect caused by said bacteria.

The invention further provides for a pharmaceutical composition for the treatment and/or prevention of a GAS invasive infection related pathologic disorder. The therapeutic composition of the invention comprises an isolated and purified peptide as described above.

It should be noted that the peptide comprised within the compositions of the invention comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferably, this peptide comprises an amino acid sequence substantially as denoted by SEQ ID NO: 32 or any functional fragment, analog or derivative thereof, which composition optionally further comprises, pharmaceutically acceptable carrier, diluent, adjuvant and/or excipient.

In a further aspect, the present invention relates to the use of an agent which induces down regulation of chemokine protease activity, for inhibiting invasive and/or non-invasive infection by Gram-positive pathogenic bacteria.

Still further, the invention provides for the use of an agent which induces down regulation of chemokine protease activity, in the preparation of a pharmaceutical composition for the treatment and/or prevention of a GAS invasive infection related pathologic disorder, which composition optionally further comprises pharmaceutically acceptable carrier, diluent, adjuvant and/or excipient.

According to one embodiment, the agent used by the invention may be a purified protective peptide comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferred peptide may comprise an amino acid sequence substantially as denoted by SEQ ID NO: 32 or any functional fragment, analog or derivative thereof.

Alternatively, as an agent which induces down regulation of chemokine protease activity the invention may use an inhibitor of a serine protease.

According to a preferred embodiment, such serine protease inhibitor may be selected from the group consisting of aprotenin, trypsin inhibitor, chemotrypsin inhibitor, plasmin inhibitor, kallikrein inhibitor, benzamidine a soybean trypsin inhibitor.

The invention further provides for a method of inhibiting an invasive and/or non-invasive infection of Gram-positive pathogenic bacteria in a mammalian subject, comprising administering to said subject an inhibitory effective amount of an agent which induces down regulation of chemokine protease activity.

Still further, the invention relates to a method of preventing and/or treating a GAS invasive infection related pathologic disorder comprising administering to a mammalian subject in need of such treatment a therapeutically effective amount of an agent which induces down regulation of chemokine protease activity.

According to one specifically preferred embodiment, the agent used by the method of the invention may a purified protective peptide comprising an amino acid sequence substantially homologous to the amino acid sequence encoded by the SilCR ORF of the sil locus at position 2985 to 2863 of the complementary strand of the genomic sequence of Group A Streptococcus (GAS) bacteria sped in GenBank accession number AF493605. Preferably, the peptide used as an agent by the methods of the invention may comprise an amino acid sequence substantially as denoted by SEQ ID NO: 32 or any functional fragment, analog or derivative thereof.

Alternatively, as an agent which induces down regulation of chemokine protease activity the methods of the invention may use an inhibitor of a serine protease. Such serine protease inhibitor may be selected from the group consisting of aprotenin, trypsin inhibitor, chemotrypsin inhibitor, plasmin inhibitor, kallikrein inhibitor, benzamidine and soybean trypsin inhibitor.

The invention will be further described on the hand of the following figures, which are illustrative only and do not limit the scope of the invention which is defined by the appended claims.

### BRIEF DESCRIPTION OF THE FIGURES

FIG. 1A-1B Identification of an Attenuated Mutant of JS95 by PTTM

1A. The structure of IS256pt and a schematic presentation of PTTM strategy. IS256pt contains left and right inverted repeats (IR_{L} and IR_{R}), transposase, spectinomycin resistant gene, aad9, a tag and E. coli origin of replication (ori). Primers A and B amplify the tags and primers C and D are used to identify the insertion site. Detection of a missing tag in the output pool allows the identification of a mutant that is unable to grow in a specific tissue. (I). IS256pts contain tags of different lengths amplified by primers A and B. (II). Tansformation of JS95 by IS256pts. (III). Recovered bacteria from different tissues. (IV). Resolution on a sequencing gel.

1B. Chromatograms of PCR amplified tags isolated from a mouse inoculated with an inoculum of ˜2×10⁶ containing 21 IS256pts mutants. One tag that is present in the skin (upper panel) is missing in the spleen (indicated by an arrow in the lower panel).

Abbreviations: lib. Mut. (a library of mutants), Inp. (input), Ou (output), Poo. lib. (pooled library), sp. (spleen), sk. (skin).

FIG. 2A-2B The Ability of JS95 and of JS95::pttm 112 to Kill Mice and to Spread into the Spleen

2A. Number of mice which survived after inoculation with high doses of ˜5×10⁸ CFU of the wild type (O JS95) or with the transposon insertion mutant (▪ JS95::pttm112).

2B. Number of CFU recovered from the spleen of mice (n=3) inoculated with doses of ˜2×10⁷ CFU of JS95 (empty bars) or with JS95::pttm112 (filled bars). Data are presented as means ± standard deviation.

Abbreviations: Surv. (survivors), T (time), h. (hours), Sp. (spleen).

FIG. 3A-8C The Genomic Organization of Sil

3A. The sil locus includes the putative TCS genes silA and silB, silC that is preceded by a combox promoter, and the two putative ABC transporters genes, silD and silE. The insertion of IS256pt in JS95::pttm112 disrupts silC. The small arrow upstream of silA represents a promoter. IS1562 and blpM-homologue (BlpM^{H}) are located upstream and downstream of sil, respectively. Bent arrows represent primers that are described in Table 2.

3B. silC and silCR are transcribed in opposite directions. The relative positions of silC and silCR transcripts are shown in the upper part. The indicated StyI and BspEI sites were used for replacement of silC by the spectinomycin resistance gene, aad9. The alignment of silC with silCR, which encodes a putative competence stimulating peptide in the M18. MGAS8232 strain, is shown in the lower part. A point mutation in JS95, at position 124 destroys the start codon of the ORF predicted by J. C. Smoot et al, [(2002) ibid.].

3C. sil is located in JS95 in a region that corresponds to section 36 of M1 chromosome. The black arrows in the M1 chromosome designate genes encoding peptides with a double-glycine leader. blpM^{T} is homologues to blpM^{H} but is truncated in M1. The bent arrows represent primers used to identify the upstream and downstream regions of sil.

Abbreviations: Ins. (insertion), Prom. (promoter).

FIG. 4A-4B Virulence of JS95 and of its Derived Mutants

4A. Survival of mice inoculated with doses of ˜5×10⁷ CFU JS95 (O), JS95::pttm112 (▪), JS95ΔsilC (▴) and JS95silB (●). The rate of mortality in JS95 mutants was significantly lower than that of JS95 wild type.

4B. Blood survival is represented by the increase in the number of CFU (multiplication factor) during the incubation of the indicated strains in whole blood. Data are presented as means ± standard deviation. Abbreviations: Surv. (survivors), T (time), h. (hours), Mult. Fac. (Multiplication factor).

FIG. 5 A 17 Amino Acid Peptide Protects Mice Challenged with a Highly Invasive GAS Strain

Mice survival after inoculation with a dose of 10⁷ CFU of the wild type NF-causing strain (JS95) was monitored in the absence and presence of the indicated amounts of the 70% pure peptide (μg). Abbreviations: Surv. (survival), T (time), d. (days).

FIG. 6 The Protective Effect of SilCR is Dose-Dependent

Survival of mice inoculated subcutaneously with either 10⁸ CFU of JS95 (♦) or with 10⁸ CFU of JS95 mixed with increasing concentrations (μg/0.1 ml) of SilCR (90% pure): 3.1 (▬), 6.2 [symbol] , 12.5 ( [symbol] ), 25 (▬), and 50 (●). Mice were observed daily post inoculation. The experiments were repeated 3 times yielding similar results. Abbreviations: Surv. (survivors), T. (time) d. (days).

FIG. 7 The Peptide Protects Mice Challenged with Different Doses of a Highly Invasive GAS Strain

Mice survival was monitored after challenge with increasing doses of the wild type NF-causing strain (JS95), in the absence and presence of the 70% pure peptide (200 μg). Abbreviations: Surv. (survival), T (time), d. (days), pep. (peptide).

FIG. 8A-8D The Peptide Inhibits Skin Necrosis in Mice Challenged with a Highly Invasive GAS Strain

8A, 8C. GAS strain (JS95) was grown in Todd-Hewitt medium supplemented with 0.2% yeast extract (THY) to midlog phase (OD₆₀₀ of 0.4). Bacteria were harvested by centrifugation and washed twice in PBS and the number of bacteria was adjusted to 10⁸ CFU per 100 μl and injected under the surface of the skin. A typical mouse lesion 24 h after challenge showing a large necrotic cutaneous area.

8B, 8D. Similar procedure was performed using the test group that was supplemented with 25 μg of highly purified peptide (90% purity).

FIG. 9 The Peptide Protects Mice Challenged with a Highly Invasive GAS Strain when Applied Before Bacterial Challenge

One group of mice was injected with 200 μg of peptide (70% pure) and than challenged 24 hours later with the bacteria (◯). The control group (□) of mice that did not receive any treatment was challenged with the bacteria at day 0. Abbreviations: Surv. (survival), T (time), d. (days), Pep. (peptide).

FIG. 10 The Peptide does not Inhibit Bacterial Growth

One thousands to 1500 CFU of GAS strain JS95 were spread on the surface of a blood-agar plate, incubated at room temperature and disks with different concentrations of antibiotic (control) or peptide (25, 50 and 200 μg) were placed on the surface. The plates were incubated at 37° C. for 16 hours. Areas with no hemolysis (N. hemo.) indicate inhibition of GAS growth by antibiotics. Area with hemolysis (hemo.) indicates GAS growth in the presence of 200 μg of the peptide.

FIG. 11A-11D The Peptide Enables Recruitment of PMN into the Lesion Caused by JS95

Haematoxylin and eosin stain of the soft-tissue lesions from a mouse challenged with 10⁸ CFU of JS95 24 h after inoculation (11A-11B) in the presence of 100 μg of 70% pure peptide (11C, 11D). After 24 hours, skin samples were stained with hematoxiline and eosine and examined by microscopy at 50 (11A, 11C) and 400 (11B, 11D) fold magnification. There is extensive necrosis of fascia, massive presence of bacteria but no infiltration of neutrophils. There were no differences in the histopathology findings as determined in two mice in 4 different experiments.

FIG. 12A-12C Gross Pathology and Histopathology of Debrided Tissue From a Patient with GAS NF of the Arm

12A. The patient described in case 1 underwent fasciotomy. The debrided tissues contained extensive necrotic fascia and subcutaneous tissues, but the underlying muscles were unaffected.

12B. Haematoxylin and eosin staining of a typical debrided section reveals extensive necrosis resulting in the separation of collagen fibers and presence of a massive amount of bacteria but no neutrophils infiltration (magnification 400×).

12C. The arm is shown several weeks after wound closure and the application of skin graft.

FIG. 13A-13C Histopathology of Soft-Tissue Derived from Mice at Different Time Points after Challenge with JS95 Strain

Haematoxylin and eosin stain of the soft-tissue lesions biopsy from a mouse challenged with 10⁸ CFU of JS95:3 (13A), 6 (13B), and 12 (13C) h after inoculation, respectively (magnification 200×).

FIG. 14 The Degradation of IL-8 by JS95 Supernatant is Modulated by SilCR

JS959 was grown in the absence and presence of SilCR (50 μg/ml). IL-8 was incubated with the indicated supernatant fractions and then subjected to 18% SDS-PAGE. The separated protein bands were electro-transferred to a nitrocellulose membrane. Anti-IL-8 antibody and a secondary antibody conjugated to horseradish peroxidase were used to detect IL-8. The number of the lane represents incubation of IL-8 with: 1, DMEM+FCS (control); 2, JS95 supernatant; 3, JS95 supernatant grown in the presence of SilCR; 4, JS95 supernatant, pretreated with pefabloc SC; 5, supernatant of JS95 grown in the absence of SilCR, then 50 μg/ml of SilCR was added to the degradation assay.

FIG. 15A-15B Degradation of Chemokines by the JS95 Supernatant

15A The column numbers represent the ELISA results after incubation of IL-8 with: 1, DMEM+FCS (control); and the supernatants of: 2, JS95; 3, JS95 grown in the presence of SilCR; 4, JS95 pretreated with aprotenin; 5, JS95 pretreated with soybean trypsin inhibitor; 6, JS95 pretreated with benzamidine. The background of the ELISA assay (0.08±0.004) was subtracted from the shown results. The data represent the mean ± SD, of three separate experiments.

15B. The dots in the control panel represent MIP-2 that was incubated in DMEM+FCS, in the absence of JS95 supernatant and spotted at the following doses: 30, 15, 7.5, 3.75 and 1.87 ng (from left to right). The dots in the lower panel were generated in the same way, except that MIP-2 was previously incubated with JS95 supernatant.

Abbreviations: cont. (control).

FIG. 16A-16C SilCR Protects Mice Against JS95-Induced Killing

16A. Shows a representative experiment of survival of mice challenged with 10⁸ (♦, ▪) or 10¹⁰ (▴) CFU of JS95 in the absence (♦) and the presence (▴, ▪) of SilCR (50 μg 90% purified). The experiment was repeated 4 times yielding similar results.

16B. Shows representative lesions in mice infected with 10⁸ CFU of M1 strain 340.

16C. mice infected with 10⁸ CFU of M1 strain 340+SilCR (50 μg 90% purified) 48 h after inoculation.

Abbreviations: Surv. (survivors, T (time) d. (days).

### DETAILED DESCRIPTION OF THE INVENTION

Definitions:

Missense mutation—A codon change that alters the amino acid encoded.

Chemokine—A family of small soluble chemotactic proteins that stimulate the directed migration and activation of leukocytes.

Pheromone peptide—Bacteria secrete pheromone molecules, which accumulate outside of the cell. In this manner, bacteria communicate with each other. In Gram-positive bacteria pheromones are small peptides that are sensed when reaching a critical concentration in the culture medium, leading to alterations in bacterial gene expression.

ELISA—Acronym for Enzyme-Linked ImmunoSorbent Assay. A quantitative method for estimating the amount of a compound using antibodies linked to an enzyme that catalyses an easily measurable color reaction.

Dot-Blot

A technique for quantifying proteins, which are spotted onto a nitrocellulose membrane and recognized by a specific antibody linked to an enzymatic detection system.

RT-PCR

A two-phase reaction for detecting messenger RNA gene transcripts. The enzyme reverse transcriptase is used to convert the RNA to cDNA, which is then amplified by polymerase chain reaction (PCR).

The inventors have identified in GAS strain JS95 the locus sil, which contributes to GAS virulence by enhancing GAS spreading and invasion of internal organs. Mutants of sil were attenuated in their ability to disseminate from the skin into the spleen and to cause a lethal infection in a mouse-model of human NF. The results of the present invention indicate that the sil locus is present in 3 different M14 virulent strains isolated from patients with NF, which were also lethal in the mouse model. Recently, the genome of an M18 type strain associated with acute rheumatic fever was sequenced [Smoot (2002) ibid.], and was shown herein by the present inventors as containing a locus highly homologous to sil. Interestingly, this locus was reported as comprising an ORF, which encodes a 41 amino acid precursor, which processed to form a 17 amino acid peptide. The corresponding silCR sequence defined by the present invention in the GAS JS95 strain contains a G to A mutation, which results in elimination of the ATG codon and therefore prevention of the 17 amino acid peptide formation. Surprisingly, this peptide was found by the present inventors to possess anti-invasive properties. Thus, sil locus and particularly the 17 amino acid peptide may regulate virulence genes in GAS strains of different genetic backgrounds causing diverse types of human diseases.

Thus, in a first aspect, the invention relates to an isolated and purified peptide capable of inhibiting an invasive and/or non-invasive infection of Gram-positive pathogenic bacteria, comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. As indicated above, the SilCR ORF encodes a putative 41 amino acids precursor peptide which is processed to form the 17 amino acid mature peptide, which is therefore encoded by the SilCR ORF at positions 2913 to 2863.

Therefore, in one particular embodiment, the invention relates to an isolated and purified protective peptide capable of inhibiting an invasive infection of Gram-positive pathogenic bacteria. The peptide of the invention comprises an amino acid sequence DIFKLVIDHISMKARKK, also denoted by SEQ ID NO: 32 or any functional fragment, analog or derivative thereof.

The homology between any peptide described by the invention or used by the methods or compositions of the present invention, and the amino acid sequence encoded by the SilCR ORF of the sil locus at position 2985 to 2863 9 and most preferably at position 2913 to 2863) of the complementary strand of the genomic sequence of Group A Streptococcus (GAS), may range between 10% to 100% homology, preferably, 20% to 90% homology, and most preferably, between 30% to 80% homology.

The terms functional derivatives and functional fragments used herein mean the peptide or any fragment thereof, with any insertions, deletions, substitutions and modifications, that inhibits the invasive infection and/or non-invasive infection of a pathogenic Gram-positive bacteria.

In a specifically preferred embodiment such bacteria may be Streptococcus sp., selected from the group consisting of Group A Streptococcus (GAS) bacteria, Group B Streptococcus bacteria and Group G Streptococcus bacteria, preferably, Group A Streptococcus (GAS) bacteria.

In yet another embodiment, the isolated peptide of the invention is capable of inhibiting the spreading of a GAS bacteria and/or tissue necrosis and lethal effect caused by this bacteria.

According to a preferred embodiment, the peptide of the invention is capable of inhibiting invasive infection that commonly may lead to any one of soft tissue infection, bacteremia, septicemia, toxic shock syndrome (TSS) and necrotizing fasciitis (NF) and to the non-invasive sequela, including rheumatic fever and acute glomerulonephritis.

In another specifically preferred embodiment, the peptide of the invention is capable of inhibiting an invasive infection caused by GAS bacteria that may be any one of Streptococcus pyogenes, Streptococcus pneumoniae, Staphylococcus aureus and oral Streptococci. Preferably, the GAS bacteria may be a virulent strain of Streptococcus pyogenes. As a non-limiting exam, such virulent may be any one of M1,-M3, M1T1, M14, JS20, JS95 and JS198.

To date, there have been limited therapeutic applications involving peptides, due in considerable part to lack of oral bioavailability and to proteolytic degradation. Typically, for example, peptides are rapidly degraded in vivo by exo- and endopeptidases, resulting in generally very short biological half-lives. Degradation of the peptides by proteolytic enzymes in the gastrointestinal tract is likely an important contributing factor. The design of peptide mimics which are resistant to degradation by proteolytic enzymes has become of increasing interest to peptide chemists. A primary goal has been to reduce the susceptibility of mimics to cleavage and inactivation by peptidases. In one approach, such as disclosed by Sherman and Spatola, [J. Am. Chem. Soc. 112:433 (1990)], one or more amide bonds have been replaced in an essentially isosteric manner by a variety of chemical functional groups. This stepwise approach has met with some success in that active analogs have been obtained. In some instances, these analogs have been shown to possess longer biological half-lives than their naturally-occurring counterparts.

In another approach, a variety of uncoded or modified amino acids such as D-amino acids and N-methyl amino acids have been used to modify mammalian peptides. Alternatively, a presumed bioactive conformation has been stabilized by a covalent modification, such as cyclization or by incorporation of ψ-lactam or other types of bridges. See, e.g., Veber and Hirschmann, et al. [Proc. Natl. Acad. Sci. USA. 75:2636 (1978); Thorsett, et al., Biochem. Biophys. Res. Comm. 111:166 (1983)]. The primary purpose of such manipulations has not been to avoid metabolism or to enhance oral bioavailability but rather to constrain a bioactive conformation to enhance potency or to induce greater specificity.

Another approach, disclosed by Rich, D. H. [in Protease Inhibitors, Barrett and Selveson, eds., Elsevier, p. 179-217 (1986)], has been to design peptide mimics through the application of the transition state analog concept in enzyme inhibitor design.

Nicolaou and Hirschmann, et al., Design and synthesis of a peptidomimetic employing β-D-glucose for scaffolding [in Peptides, Chemistry, Structure and Biology: Proceedings of the 11th American Peptide Symposium, Rivier and Marshall, eds., ESCOM (1990), p. 881-884].

Further peptidomimetics are compounds that appear to be unrelated to the original peptide, but contain functional groups positioned on a non-peptide scaffold that serve as topographical mimics. This type of peptidomimetics is referred to herein as a “non-peptidyl analogue”. Such peptidomimetics may be identified using library screens of large chemical databases.

Peptidomimetics (both peptide and non-peptidyl analogues) may have improved properties (e.g., decreased proteolysis, increased retention or increased bioavailability). Peptidomimetics generally have improved oral availability, which makes them especially suited to treatment of different pathological conditions. It should be noted that peptidomimetics may or may not have similar two-dimensional chemical structures, but share common three-dimensional structural features and geometry. Each peptidomimetic may further have one or more unique additional binding elements.

As discussed above, the lack of structure of linear peptides renders them vulnerable to proteases in human serum and acts to reduce their affinity for target sites, because only few of the possible conformations may be active. Therefore, it is desirable to optimize the peptide structure, for example by creating different derivatives of the various peptides of the invention.

Thus, the peptide of the invention or any fragment, analog or derivatives thereof may be in the form of a dimer, a multimer or in a spatially constrained conformation. The peptide of the invention may be conformationally constrained by internal bridges, short-range cyclization, extension or other chemical modification.

In order to improve peptide structure, the peptides of the invention can be coupled through their N-terminus to a lauryl-cysteine (LC) residue and/or through their C-terminus to a cysteine (C) residue, or to other residue/s suitable for linking the peptide to adjuvants for immunization, as will be described in more detail hereafter.

The peptides of the invention, as well as derivatives thereof may all be positively charged, negatively charged or neutral.

Further, the peptides of the invention may be extended at the N-terminus and/or C-terminus thereof with various identical or different amino acid residues. As an example for such extension, the peptide may be extended at the N-terminus and/or C-terminus thereof with identical or different hydrophobic amino acid residue/s, which may be naturally occurring or synthetic amino acid residue/s. A preferred synthetic amino acid residue may be D-alanine.

An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond.

Another example is the incorporation of an N-terminal lysyl-fatty acyl tail, the lysine serving as linker and the fatty acid as a hydrophobic anchor. A suitable fatty acid may be palmitic acid.

In addition the peptides may be extended by aromatic amino acid residue/s, which may be naturally occurring or synthetic amino acid residue/s. A preferred aromatic amino acid residue may be for example, tryptophan.

Further, according to the invention, the peptides of the invention may be extended at the N-terminus and/or C-terminus thereof with various identical or different organic moieties, which are not a naturally occurring or synthetic amino acids. As an example for such extension, the peptide may be extended at the N-terminus and/or C-terminus thereof with an N-acetyl group.

These extended peptides, as other peptides of the invention, can also be used for both a treatment of acute toxic shock and NF, or any other pathologic disorder caused by an invasive and/or non-invasive infection and of the harmful effects caused thereby and for preventing the invasive infection by virulent invasive bacterial pathogens.

The peptides of the invention may be further optimized by scanning of combinatorial libraries of peptides and libraries of cyclic peptidomimetics.

It is to be appreciated, that any substance that can mimic the structure, sequence or function of the peptide used by the invention, is contemplated within the scope of the invention.

In a second aspect, the invention relates to a method of inhibiting an invasive infection of Gram-positive pathogenic bacteria in a mammalian subject, comprising administering to said patient an inhibitory effective amount of an isolated and purified peptide or of a composition comprising the same, which peptide comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. It should be noted that the SilCR ORF encodes the putative 41 amino acid precursor peptide, which is processed to form the preferred 17 amino acids peptide of the invention. Preferably, this peptide comprises an amino acid sequence substantially as denoted by SEQ ID NO: 32 or any functional fragment, analog or derivative thereof.

According to one embodiment of this aspect, the Gram-positive pathogenic bacteria may be Streptococcus sp., such as Group A Streptococcus (GAS) bacteria, Group B Streptococcus bacteria or Group G Streptococcus bacteria, preferably, Group A Streptococcus (GAS) bacteria.

In yet another embodiment, the peptide used by the method of the invention is capable of inhibiting the spreading of GAS bacteria and/or tissue necrosis and lethal effect caused by said bacteria. More specifically, this method utilizes the peptide as defined by the invention herein before.

Still further, the invention relates to a method of preventing and/or treating a GAS invasive infection related pathologic disorder. According to one embodiment, this method of treatment comprises administering to a mammalian subject in need of such treatment a therapeutically effective amount of an isolated and purified peptide or of a composition comprising the same. The peptide used by this method comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferably, the peptide used by this method, comprises an amino acid sequence substantially as denoted by SEQ ID NO: 32 or any functional fragment, analog or derivative thereof.

In a specifically preferred embodiment, the method of the invention is intended for the treatment and/or prevention of a GAS invasive infection related pathologic disorder. Such disorder may be as a non-limiting example, a soft tissue infection, bacteremia, septicemia, toxic shock syndrome (TSS) and necrotizing fasciitis (NF), and non-invasive infection that may lead to rheumatic fever or acute glomerulonephritis.

According to a particular embodiment of the method of the invention, the effective amount of the isolated and purified peptide or of a composition comprising the same may be administered to a subject in need of such treatment, prior to potential exposure to said pathogenic bacteria.

The term ‘effective amount’ for purposes herein is that determined by such considerations as are known in the art. The amount must be sufficient to inhibit or prevent invasive infection by pathogenic bacteria. More specifically, the amount must be sufficient to prevent bacterial spreading and tissue necrosis.

The peptide of the invention may be administered directly by the method of the invention, to the subject to be treated or, depending on the size of the compound, it may be desirable to conjugate them to a carrier prior to their administration. Therapeutic formulations may be administered in any conventional dosage formulation. Formulations typically comprise at least one active ingredient, as defined above, together with one or more acceptable carriers thereof.

The magnitude of therapeutic dose of the composition of the invention will of course vary with the group of patients (age, sex, etc.), the nature of the condition to be treated and with the route administration and will be determined by the attending physician. More specifically, the effective amount of said isolated and purified peptide or composition comprising the same may be administered to a subject in need of such treatment, in a single dose.

Alternatively, the effective amount of said isolated and purified peptide or composition comprising the same may be administered to said subject in multiple doses.

The effective amount of the isolated and purified peptide or composition comprising the same may be administered by the method of the invention, to a subject in need of such treatment, by a single route of administration.

In yet another embodiment, the effective amount of the isolated and purified peptide or composition comprising the same may be administered to a subject in need of such treatment by at least two different routes of administration.

Preferably, the peptide or the composition of the invention may be administered through intravenous, parenteral, transdermal, subcutaneous, intravaginal, intranasal, mucosal, sublingual, topical and rectal administration and any combinations thereof.

More preferably, the effective amount of said isolated and purified peptide or composition comprising the same may be administered to said subject subcutaneously.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi. The prevention of the action of microorganisms can be brought about by various antibacterial and antifungal agents, for example, parabens, chlorobutanol, phenol, sorbic acid, thimerosal, and the like. In many cases, it sill be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

Oral administration is expected to be less effective, particularly where the active compound is a peptide, because the peptide may be digested before being taken up. Of course, this consideration may apply less to an active peptide invention which is modified as described above, e.g., by being cyclic peptide, by containing non-naturally occurring amino acids, such as D-amino acids, or other modification which enhance the resistance of the peptide to biodegradation Decomposition in the digestive tract may be lessened by use of certain compositions, for instance, by confining the active compounds comprised in the compositions of the invention in microcapsules such as liposomes. The pharmaceutical composition may be also provided in the form of a suppository, nasal spray or sublingual tablet. The dosage of the peptide of the invention may depend upon the condition to be treated, the patient's age, body weight, and the route of administration, and will be determined by the attending physician.

It should be noted that the uptake of an active peptide, may be facilitated by a number of methods. For instance, a non-toxic derivative of the cholera toxin B subunit, or of the structurally related subunit B of the heat-labile enterotoxin of enterotoxic E. coli may be added to the composition, see U.S. Pat. No. 5,554,378.

According to a specifically preferred embodiment, an effective amount or dose of the peptide of the invention may range between 0.5 μg/kg to 100 mg/kg of body weight. Preferably, an effective amount may range between 10 μg/kg to 10 mg/kg of body weight, most preferably, between 300 μg/kg to 5 mg/kg of body weight.

According to a specifically preferred embodiment, the method of the invention is intended for the treatment of a human patient. Although the method of the invention is particularly intended for the treatment of GAS invasion related diseases in humans, other mammals are included. By way of non-limiting examples, mammalian subjects include monkeys, equines, cattle, canines, felines, rodents such as mice and rats, and pigs.

It should be appreciated that the present invention further provides for a composition, which inhibits the invasive and non-invasive infection of Gram-positive pathogenic bacteria. The inhibitory composition of the invention comprises as an active ingredient an isolated and purified peptide, in an effective amount to inhibit spreading, tissue necrosis and/or lethal effect caused by said bacteria. The peptide comprised within the composition of the invention comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferably, this peptide comprises an amino acid sequence substantially as denoted by SEQ ID NO: 32 or any functional fragment, analog or derivative thereof, which composition optionally further comprises pharmaceutically acceptable carrier, diluent, adjuvant and/or excipient.

In one embodiment, the composition of the invention, inhibits and/or prevents the invasive and non-invasive infection of Streptococcus sp., such as GAS, group B streptococci or group G streptococci particularly GAS. More particularly, such GAS bacteria may be any one of Streptococcus pyogenes, Streptococcus pneumoniae, Staphylococcus aureus and oral Streptococci and most particularly, Streptococcus pyogenes.

According to a specifically preferred embodiment, the peptide comprised within the composition of the invention is capable of inhibiting the spreading of a GAS bacteria and/or tissue necrosis and lethal effect caused by said bacteria.

The invention further provides for a pharmaceutical composition for the treatment and/or prevention of a GAS invasive infection related pathologic disorder. The therapeutic composition of the invention comprises an isolated and purified peptide as described herein before. Preferably, this peptide comprises an amino acid sequence substantially as denoted by SEQ ID NO: 32 or any functional fragment, analog or derivative thereof, which composition optionally further comprises pharmaceutically acceptable carrier, diluent, adjuvant and/or excipient.

According to a specifically preferred embodiment, the therapeutic composition of the invention may be particularly suitable for the treatment and/or prevention of a GAS invasive infection related pathologic disorder such as for example, soft tissue infection, bacteremia, septicemia, toxic shock syndrome (TSS) and necrotizing fasciitis (NF) and non-invasive sequela such as rheumatic fever and acute glomerulonephritis.

In a specifically preferred embodiment, the therapeutic composition provided by the invention may be used for the treatment and/or prevention of a pathologic disorder caused by a virulent strain of Streptococcus pyogenes.

The peptides or any fragments, analogs or derivatives thereof comprised as an active ingredient within the compositions of the invention, may be any peptides according to the invention, e.g., in the form of a monomer, dimer, a multimer or in a constrained conformation, as well as the other modifications described above.

The pharmaceutical composition used by the methods of the invention can be prepared in dosage unit forms and may be prepared by any of the methods well-known in the art of pharmacy. In addition, the pharmaceutical compositions used by the invention may further comprise pharmaceutically acceptable additives such as pharmaceutical acceptable carrier, excipient or stabilizer, and optionally other therapeutic constituents. Naturally, the acceptable carriers, excipients or stabilizers are non-toxic to recipients at the dosages and concentrations employed.

The pharmaceutical compositions of the invention may generally contain salts, preferably in physiological concentration, such as PBS (phosphate-buffered saline), or sodium chloride (0.9% w/v), and a buffering agent, such as phosphate buffer in the above PBS. The preparation of pharmaceutical compositions is well known in the art, see e.g., U.S. Pat. Nos. 5,736,519, 5,733,877, 6,554,378, 5,439,688, 5,418,219, 5,354,900, 5,298,246, 5,164,372, 4,900,549, 4,755,383, 4,639,435, 4,457,917 and 4,064,236. The active ingredient of the pharmaceutical compositions of the present invention, which is peptide, or pharmacologically acceptable salts thereof, may preferably mixed with an excipient, carrier, diluent, and optionally, a preservative or the like pharmacologically acceptable vehicles as known in the art, see e.g., the above US patents. Examples of excipients include glucose, mannitol inositol, sucrose, lactose, fructose, starch, corn starch, microcrystalline cellulose, hydroxypropylcellulose, hydroxypropylmethyl-cellulose, polyvinylpyrrolidone and the like. Optionally, a thickener may be added, such as a natural gum, a cellulose derivative, an acrylic or vinyl polymer, or the like.

The pharmaceutical composition may be provided in solid, liquid or semi-solid form. A solid preparation may be prepared by blending the above components to provide a powdery composition. Alternatively, the pharmaceutical composition may be provided as lyophilized preparation. The liquid preparation is provided preferably as aqueous solution, aqueous suspension, oil suspension or microcapsule composition. A semi-solid composition may be provided preferably as hydrous or oily gel or ointment.

A solid composition may be prepared by mixing an excipient with a solution of the active agent comprised in the composition of the invention, gradually adding a small quantity of water, and kneading the mixture. After drying, preferably in vacuo, the mixture is pulverized. A liquid composition may be prepared by dissolving, suspending or emulsifying the active compound in water, a buffer solution or the like. An oil suspension may be prepared by suspending or emulsifying the active compound in an oleaginous base, such as sesame oil, olive oil, corn oil, soybean oil, cottonseed oil, peanut oil, lanolin, petroleum jelly, paraffin, Isopar, silicone oil, fatty acids of 6 to 30 carbon atoms or the corresponding glycerol or alcohol esters.

A composition may be prepared as a hydrous gel, e.g. for transnasal administration. A hydrous gel base is dissolved or dispersed in aqueous solution containing a buffer, and the said active agent, and the solution warmed or cooled to give a stable gel.

The pharmaceutical compositions used by the methods of the invention generally comprise a buffering agent, an agent which adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

The compositions used by the methods of the invention may further contra a pharmaceutically acceptable carrier, additive, diluent or excipient. Suitable carriers include, e.g., saline phosphate buffered saline, and saline with 5% HSA. Other suitable carriers are well known to those of skill in the art and are not a limitation on the present invention. Similarly, one of skill in the art may readily select other desired components for inclusion in a pharmaceutical composition of the invention, and such components are not a limitation of the present invention.

Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

The invention further relates to a method for disinfecting an environment and/or preventing infection caused by Gram positive bacteria, comprising the step of applying a sufficient amount of a peptide of the invention which comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605, or a composition comprising the same, onto a surface of medical equipment, medical devices and disposables, for example catheters, intravascular devices, prosthetic devices, intraluminal devices.

It is to be appreciated that the peptide of the invention may be further used for reducing and preventing infection, by applying said peptide on a surface of instruments and equipment used for invasive or non-invasive procedures performed on a patient.

In a further aspect, the invention relates to the use of an isolated and purified peptide comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605 or any functional fragment, analog or derivative thereof, as an agent for inhibiting the invasive and non-invasive infection of Gram-positive pathogenic bacteria. Preferably, such peptide comprises an amino acid sequence substantially as denoted by SEQ ED NO: 32 or any functional fragment, analog or derivative thereof.

The invention further provides for the use of the peptide of the invention, in the preparation of a composition, which inhibits the invasive infection of Gram-positive pathogenic bacteria. Such composition comprises as an active ingredient an isolated and purified peptide, in an amount effective to inhibit spreading, tissue necrosis and/or lethal effect caused by said bacteria, wherein said peptide comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferably, this peptide comprises an amino acid sequence substantially as denoted by SEQ ID NO: 32 or any functional fragment, analog or derivative thereof, which composition optionally further comprises pharmaceutically acceptable carrier, diluent, adjuvant and/or excipient.

Still further, the invention relates to the use of a peptide as defined by the invention, in the preparation of a pharmaceutical composition for the treatment and/or prevention of a GAS invasive infection related pathologic disorder. This composition comprises an isolated and purified peptide comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferably, this peptide comprises an amino acid sequence substantially as denoted by SEQ ID NO: 32 or any functional fragment, analog or derivative thereof, which composition optionally further comprises pharmaceutically acceptable carrier, diluent, adjuvant and/or excipient.

According to a specific embodiment, the invention relates to the use of such peptide in the preparation of a composition for the treatment of a GAS invasive and/or non-invasive infection related pathologic disorder, such as soft tissue infection, bacteremia, septicemia, toxic shock syndrome (TSS) and necrotizing fasciitis (NF), and the non-invasive sequela such as rheumatic fever and acute glomerulonephritis.

The invention further relates to the peptide of the invention for use in the inhibition of invasive infection of Gram-positive pathogenic bacteria.

Still further, the invention relates to a peptide as described above, for use in the treatment and/or prevention of a GAS invasive and non-invasive infection related pathologic disorder. Such disorders may be for example, an invasive disorder such as soft tissue infection, bacteremia, septicemia, toxic shock syndrome (TSS) and necrotizing fasciitis (NF), and non-invasive sequela such as rheumatic fever and acute glomerulonephritis.

As shown by Examples 8 to 10, the findings of the present invention demonstrate for the first time, the existence of a novel virulence trait in GAS that prevents bacterial phagocytosis by interfering with host chemokine functions. A paucity of neutrophil migration to the skin and subcutaneous tissues has been reported in cases of human NF [Cockerill, F. R. Clin. Infect. Dis. 26(6):1448-58 (1998); Norrby-Teglund, A. et al. J. Infect. Dis. 184(7):853-60 (2001)]. Without being bound by any theory, the inventors suggest that due to degradation of IL-8 and retardation of neutrophil recruitment, the bacteria can multiply and rapidly spread from the site of inoculation to surrounding tissues, resulting in necrosis of fascia, skin and underlying tissues including blood vessels. Indeed, inventors have found many thrombosed and infarcted blood vessels in Case 2. Through the infarcted endothelium the bacteria might initiate an invasive systemic infection. The presence of vessel thrombosis with soft-tissue infarction is a characteristic pathologic finding in human NF [Barker (1987) ibid.]. Furthermore, in the baboon model of GAS NF there was an intense neutrophil influx into the site of inoculation in surviving baboons, whereas nonsurvivors exhibited no influx of neutrophils, intravascular coagulation and extensive bacterial colonization [Taylor, F. B. et al. Clin. Infect. Dis. 29(1):167-77 (1999]. After reaching the circulation through the infarcted endothelium, GAS like Staphylococcus aureus [Gresham, H. D. et al. J. Immunol. 164(7):3713-22 (2000)] are phagocytised and some survive within neutrophils. This perhaps could facilitate GAS spreading to deeper tissue and organs [Medina, E. et al. J. Infect. Dis. 187(4):597-603 (2003)].

To retard neutrophil influx GAS also produces an extracellular C5a peptidase, which cleaves the human neutrophil chemoattractant C5a [Wexler, D. E. et al. Proc. Natl. Acad. Sci. U.S.A. 82(23):8144-8 (1985); Ji, Y. et al. Infect. Immun. 64(2):503-10 (1996)]. However, the inventors did not observe any change in the amount of surface expressed C5a peptidase in the absence and presence of SilCR (not shown). GAS also produces the broad-spectrum cysteine protease SpeB, which plays a complicated role in pathogenesis through degradation of host tissue proteins and immunoglobulins [Lukomaki, S. et al. Infect. Immun. 67(4):1779-88 (1999); von Pawel-Rammingen, U. Curr. Opin. Microbiol. 6(1):50-5 (2003)], while simultaneously autodegrading bacterial virulence factors such as M protein and secreted superantigens [Kansal, R. G. et al. J. Infect. Dis. 187(3):398-407 (2003)]. The inventors have found that an isogenic SpeB-negative mutant of GAS M1 strain 5448 degraded IL-8 equally to the parent strain (not shown). These results indicate that probably neither the GAS C5a peptidase nor the cysteine protease SpeB are responsible for IL-8 degradation.

The therapeutic effect of SilCR may be exerted through its interaction with the bacterium, as SilCR did not stimulate neutrophil recruitment itself nor did it protect mice when simultaneously administered at a site distant to the location of bacterial challenge.

Because SilCR improved neutrophil recruitment in mice challenged with serotype M14, M1 and M3 strains, a sensor system capable of interacting with the SilCR pheromone peptide may be widely distributed among different GAS strains. The absence (M1 and M3) or mutation (M14) of the gene encoding the SilCR pheromone in those GAS strains may eliminate a counter-regulatory mean to a GAS chemokine proteolysis, resulting in an increased virulence potential for invasive soft tissue infections.

The discovery of the action of SilCR constitutes the first report of a GAS signaling molecule that modulates the pathogens virulence potential. The effect of SilCR is manifested at a most critical point of GAS interaction with the host innate immune response, namely phagocytosis and killing by neutrophils. The results of the present invention also represent the first demonstration of the therapeutic potential of a bacterial pheromone peptide in treatment of an infectious disease condition, in a sense giving GAS “a taste of its own medicine”. Deciphering of the mechanisms of action of SilCR may shed light on GAS pathogenesis and offers new therapeutic options for invasive GAS infections such as NF.

Therefore, in a further aspect the present invention relates to the use of an agent which induces down regulation and/or inhibition of chemokine protease activity, for inhibiting invasive and/or non-invasive infection by Gram-positive pathogenic bacteria.

Still further, the invention provides for the use of an agent which induces down regulation and/or inhibition of chemokine protease activity, in the preparation of a pharmaceutical composition for the treatment and/or prevention of a GAS invasive infection related pathologic disorder, which composition optionally further comprises pharmaceutically acceptable carrier, diluent, adjuvant and/or excipient.

According to one embodiment, the agent used by the invention may be a purified protective peptide comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferred peptide may comprise an amino acid sequence substantially as denoted by SEQ ID NO: 32 or any functional fragment, analog or derivative thereof.

Alternatively, as an agent, which induces down regulation and/or inhibition of chemokine protease activity, the invention may use an inhibitor of a serine protease.

According to a preferred embodiment, such serine protease inhibitor may be selected from the group consisting of aprotenin, trypsin inhibitor, chemotrypsin inhibitor, plasmin inhibitor, kallikrein inhibitor, benzamidine and soybean trypsin inhibitor.

The invention further provides for a method of inhibiting an invasive and/or non-invasive infection of Gram-positive pathogenic bacteria in a mammalian subject, comprising administering to said subject an inhibitory effective amount of an agent, which induces down regulation and/or inhibition of chemokine protease activity.

Still further, the invention relates to a method of preventing and/or treating a GAS invasive infection related pathologic disorder comprising administering to a mammalian subject in need of such treatment a therapeutically effective amount of an agent, which induces down regulation and/or inhibition of chemokine protease activity.

According to one specifically preferred embodiment, the agent used by the method of the invention may a purified protective peptide comprising an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605. Preferably, the peptide used as an agent by the methods of the invention may comprise an amino acid sequence substantially as denoted by SEQ ID NO: 32 or any functional fragment, analog or derivative thereof.

Alternatively, the methods of the invention may use as an agent which induces down regulation and/or inhibition of chemokine protease activity an inhibitor of a serine protease. Such serine protease inhibitor may be selected from the group consisting of aprotenin, trypsin inhibitor, chemotrypsin inhibitor, plasmin inhibitor, kallikrein inhibitor, benzamidine and soybean trypsin inhibitor.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an”, and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Bacterial Strains and Plasmids

The bacterial strains and plasmids used in this study are listed in Table 1. GAS strains were grown in Todd-Hewitt medium supplemented with 0.2% yeast extract (THY) and E. coli was grown in Luria broth (LB). Antibiotics used for GAS: 250 μg ml⁻¹ kanamycin, 50 μg ml⁻¹ spectinomycin, 500 μg ml⁻¹ streptomycin, and 1 μg ml⁻¹ erythromycin and for E. coli: 100 μg ml⁻¹ ampicillin, 50 μg ml⁻¹ spectinomycin, and 750 μg ml⁻¹ erythromycin.

A Murine Model of Invasive Soft-Tissue Infection

The ability of GAS to cause lethal infection in ten-gram BALB/c female mice (Harlan Laboratories, Jerusalem, Israel) was assessed as described before [Ravins, M. et al. J. Infect. Dis. 182:1702-11 (2000)]. Briefly, 10⁸ CFU of log-phase bacteria were washed twice in PBS, resuspended in 0.1 ml PBS and injected subcutaneously into the back of a mouse. The number of CFU injected was verified for each experiment by plating bacteria on THY-blood agar plates and counting. Each group contained 8 to 10 mice that were observed daily for death and changes in the lesions' properties such as size and depth, or were sacrificed at different times after inoculation for histopathological assessment. To determine the presence of bacteria in skin and spleen, mice were sacrificed; a wide area surrounding the skin lesions was excised, spleens were removed, and organs were homogenized in 10 or 1 ml of sterile PBS, respectively. Aliquots were plated and streptococcal colonies were counted. The Institutional Ethics Committee for animal care approved all animal procedures (approval number MD 79.17-4).

DNA Manipulation and Primers

All manipulations of chromosomal and plasmid DNA were performed by standard techniques [Sambrook, J. et al. Cold Spring Harbor Laboratory Press (1989)]. The primers used are listed in Table 2. Cloning and primers were designed using Vector NTI software (InforMax, Inc. Bethesda, Md.). Alignment analyses were done by BLAST <http://www.ncbi.nlm.nih.gov/BLAST> and FASTA (GCG, University of Wisconsin).

The emm typing was conducted according to R. Facklam et al, [Emerg Infect Dis, 5:247-253 (1999)] and confirmed by the National Centers for Disease Control, Biotechnology Core Facility Computing Laboratory http://www.cdc.gov/ncidod/biotech/strep/strepblast.html

TABLE 1
Bacterial strains and plasmids.

| Strain/ | | Reference/ |
|---|---|---|
| plasmid | Description | source |
| JS95 | GAS strain isolated | A. E. Moses et al, |
| | from a patient with | Emerg Infect Dis, |
| | NF | 8: 421-426 (2002) |
| JS95::pttm112 | silC PTTM-inactivated | The present invention |
| | derivative of JS95 | |
| JS95ΔsilC | silC deletion mutant | The present invention |
| | of JS95 | |
| JS95silB- | silB insertional- | The present invention |
| | inactivated | |
| | derivative of JS95 | |
| JS95silD- | silD insertional- | The present invention |
| | inactivated | |
| | derivative of JS95 | |
| JS95Δemm14 | emm14 deletion mutant | The present invention |
| | of JS95 | |
| pMGC57 | A plasmid containing | W. R. Lyon et al, |
| | IS256 with a unique | EMBO J. 17: 6263-6275 |
| | EcoRV si | (1998) |
| pGEM-T-Easy | Cloning vector | Promega, Madison, |
| | | Wisconsin |
| pUC19 | Cloning vector | New England Biolabs, |
| | | Inc. Beverly, MA |
| pCHOP-10 | pCRII (Invitrogen, | A gift of Dr. Y. Dor |
| | Carlsbad, California) | (The Hebrew University, |
| | containing 677 bp of | Jerusalem, Israel) |
| | the Mouse chop-10 mRNA | |
| pFW11 | A streptococcal shuttle | A. Podbielski et al, |
| | vector containing the | Gene, 177: 137-47 |
| | aad9 spectinomycin | (1996) |
| | resistance gene | |
| pIS256pts | Plasmids containing | The present invention |
| | tags of different | |
| | lengths | |
| pORI280 | vector for gene | K. Leenhouts et al, |
| | replacement in GAS | Mol Gen Genet, 253: |
| | | 217-24 (1996) |
| pJRS233 | Streptococcus-E-coli | J. Perez-Casal et al, |
| | temperature sensitive | Mol Microbiol, 8: |
| | shuttle vevector | 809-19 (1993) |

TABLE 2

PCR and sequencing primers.

| | | | | |
|---|---|---|---|---|
| | | SEQ. | | |
| | | ID. | | |
| Name | Sequence | NO. | Description/Location | Reference |

| | | | | |
|---|---|---|---|---|
| Universal | 5′-GTAAAAAACGACGGCCAGT-3′ | 1 | M13/pUC sequencing primer | New England Biolabs, |
| | | | (−20) | Inc. Beverly, MA |
| Reverse (A) | 5′-AACAGCTATGACCATG-3′ | 2 | M13/pUC reverse sequencing | New England Biolabs, |
| | | | primer (−21) | Inc. Beverly, MA |
| | | | Forward primer for | |
| | | | tag amplification | |
| B | 5′-AGCAGTTCGTAGTTATCTTG-3′ | 3 | Reverse primer for | The invention |
| | | | tag amplification | |
| C | 5′-TTATCAGCAATAAACGAGC-3′ | 4 | Inverse PCR primer | The invention |
| | | | from IR_{R} | |
| D | 5′-AAAGTCCTCCTGGGTATG-3′ | 5 | Inverse PCR primer | The invention |
| | | | from IR_{L} | |
| E | 5′-TTTGGCAGCTTTGACGATGG-3′ | 6 | Inverse PCR primer | The invention |
| | | | from 3′ of silE | |
| F | 5′-TCTTCAAGCAGCTGATTGGG-3′ | 7 | Inverse PCR primer | The invention |
| | | | from 5′ of silA | |
| SilB-f1 | 5′-GGAGTTGGTTTATCAAATGTCAG-3′ | 8 | 2598-2620 in sil | The invention |
| SilD-r1 | 5′-ATCTGCCACAAAGACTGATCAAG-3′ | 9 | 3213-3235 In sil | The invention |
| SilB-f2 | 5′-TTATTGGATCGGAACTTACGC-3′ | 10 | 2013-2033 in sil | The invention |
| SilD-r2 | 5′-TGCTTCCCAACAACTTACCAC-3′ | 11 | 3554-3574 in sil | The invention |
| SilB-f3 | 5′-GCTCGCTATAGTAAGCAAATCG-3′ | 12 | 2088-2109 in sil | The invention |
| SilE-r | 5′-CAGCGATTAAGCATTGAC-3′ | 13 | 5871-5888 in sil | The invention |
| HK-f | 5′-ACGAAAGGTCAATGGTTCAC-3′ | 14 | 1616-1634 in sil | The invention |
| HK-r | 5′-AGGTATGGATAAGCGTTGAG-3′ | 15 | 2338-2357 in sil | The invention |
| ABC1-f | 5′-ATGACACTTGTTACACGTCC-3′ | 16 | 3120-3138 in sil | The invention |
| ABC1-r | 5′-ACTAGTCAGCTTGACGAACTTC-3′ | 17 | 3873-3894 in sil | The invention |
| Primer1 | 5′-TATTCGCTTAGAAAATTAA-3′ | 18 | emm typing forward primer | A. M. Whatmore et al, |
| | | | | Mol Microbiol, 11: |
| | | | | 363-74 (1994) |
| Primer2 | 5′-GCAAGTTCTTCAGCTTGTTT-3′ | 19 | emm typing reverse primer | A. M. Whatmore et al, |
| | | | | Mol Microbiol, 11: |
| | | | | 363-74 (1994) |
| aad9-f | 5′ CCATGGTCCTCGAGCTCTAGATGTTAAG 3′ | 20 | aad9 forward primer | The invention |
| aad9-r | 5′ CTGGCAGGCGCTTACCAATTAGAATG 3′ | 21 | aad9 reverse primer | The invention |
| BlpM-f | 5′-TCGATATGGAGATAAAGAAACTGG-3′ | 22 | 6878-6896 in J895 sil, | The invention |
| | | | 5096-5119 in M1 | |
| SPy04B8-r | 5′-AACAGTGCTTTCAGGAACTCCT-3′ | 23 | 6804-6826 in M1 Section 36 | The invention |
| MutR-r | 5′-CTAGGTGCAATTGAGGAGTCAA-3′ | 24 | 10031-10052 in M1 section | The invention |
| | | | 36 | |
| IS1562-r | 5′-TCCTCGCACTGTTCCAATAG-3′ | 25 | 20-43 in J895 all, | The invention |
| | | | 7287-7306 section | |
| | | | 152 in M1 | |
| 8Py0479-f | 5′-AGGTGGTGTTGGAGCAGGTA-3′ | 26 | 3560-3599 in M1 section 36 | The invention |
| era-f | 5′-AAGAAGTGGTCCCAATTTCTG-3′ | 27 | 1545-1666 in M1 section 36 | The invention |
| allM-f | 5′-CGTGAAAATGAGGATCCTTCCTAAAAAACG-3′ | 28 | Forward all M primer with | The invention |
| | | | BamHI site | |
| allM-r | 5′-GGGGGCTGCAGAGCTTAGTTTTCTTCTTTGCG-3′ | 29 | Reverse all M primer with | C. Berkower et al, |
| | | | PstI site | Mol Microbiol, 31: |
| | | | | 1463-75 (1999) |
| All | 5′-GATTCCAGAAGCGATTATTG-3′ | 30 | 3′ of mga | (Podbislski, 1993) |
| C5 | 5′-AATGGCAAGTTTATCAAATGG | 31 | Leader peptide region of | A. Podbislski, Mol |
| | | | sepA | Osa Gsnat, 237: |
| | | | | 287-300 (1993) |
| | | | | (Podbislski, 1993) |

PTTM
A. Construction of pIS256pts

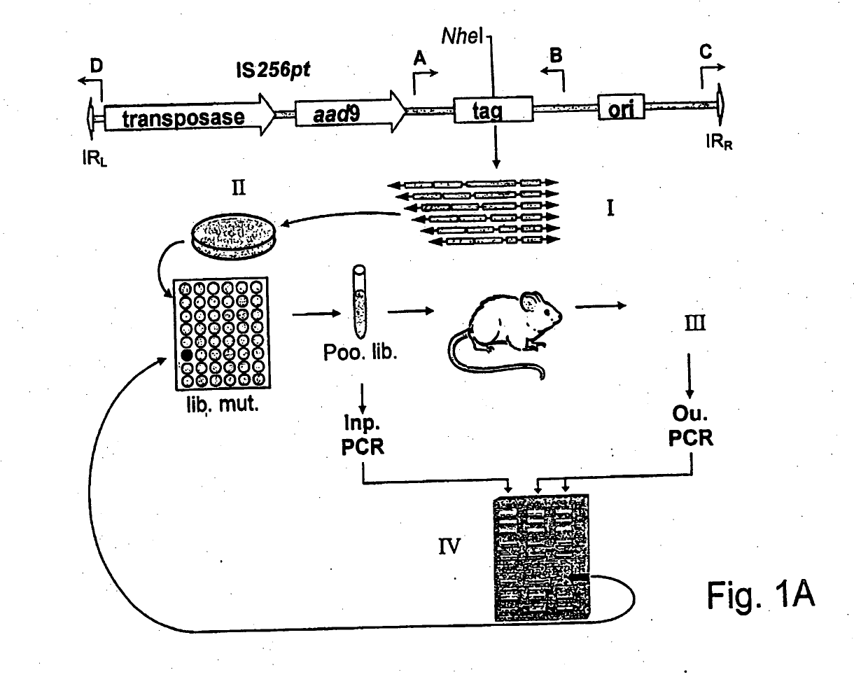

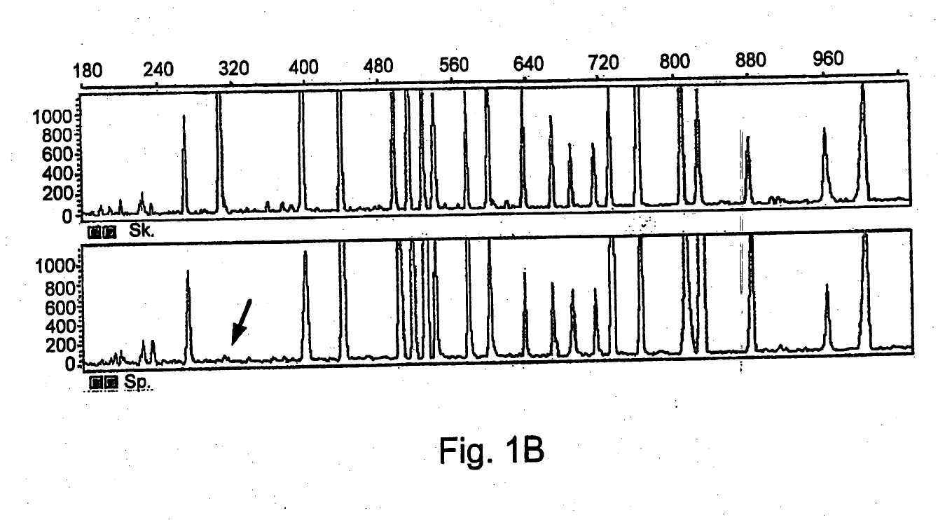

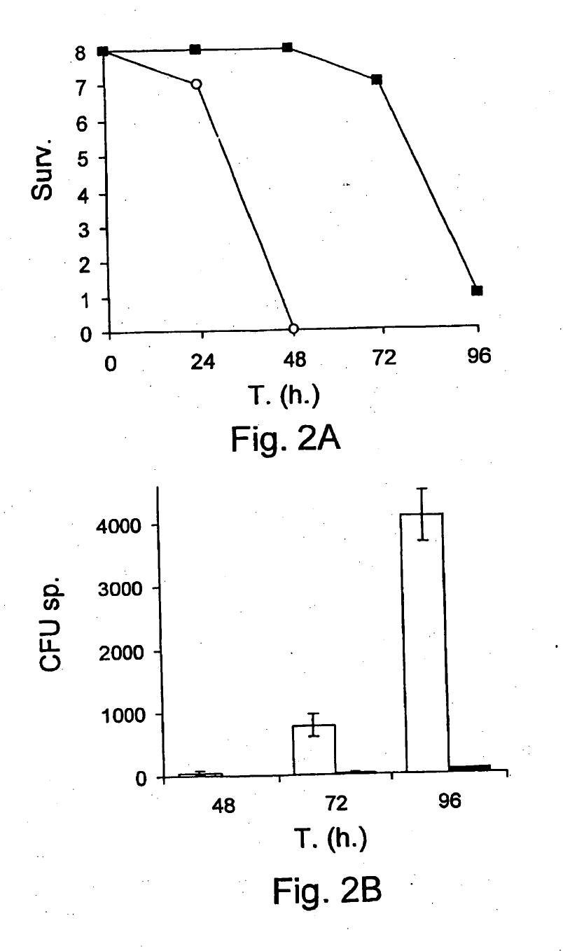

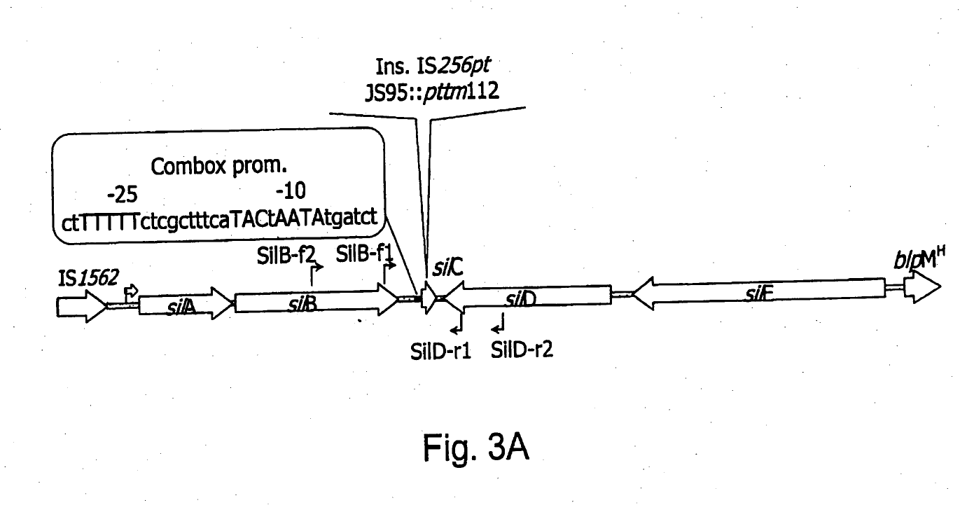

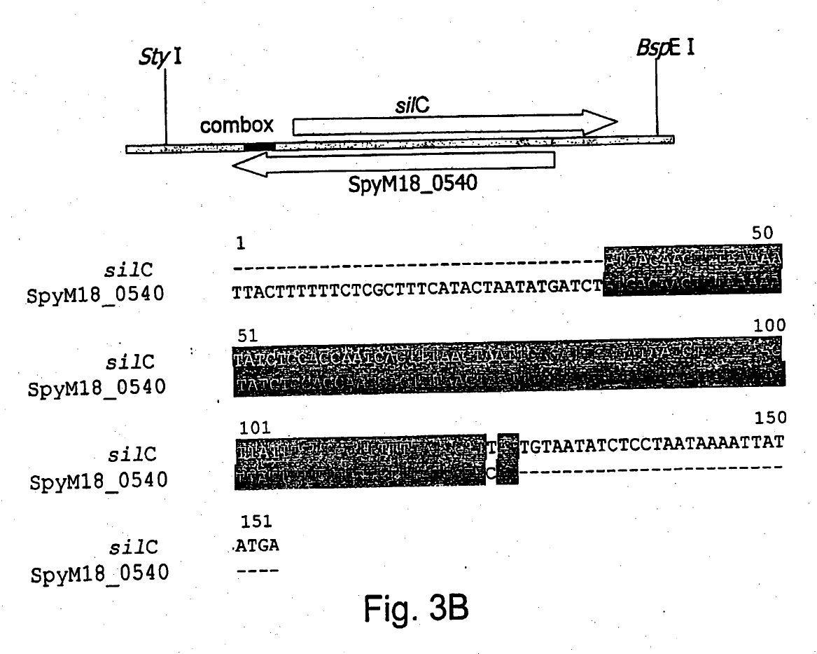

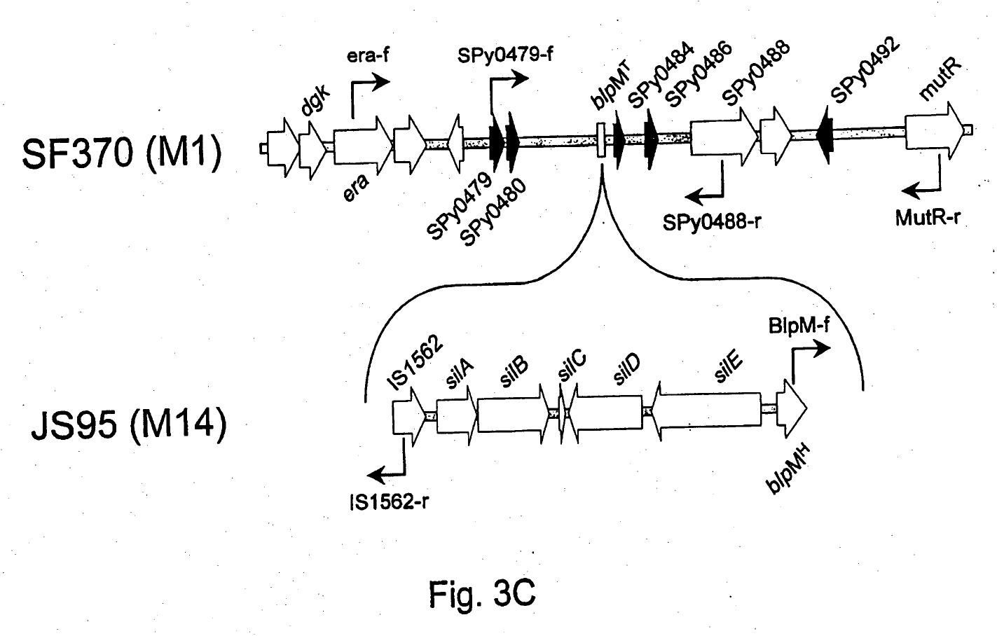

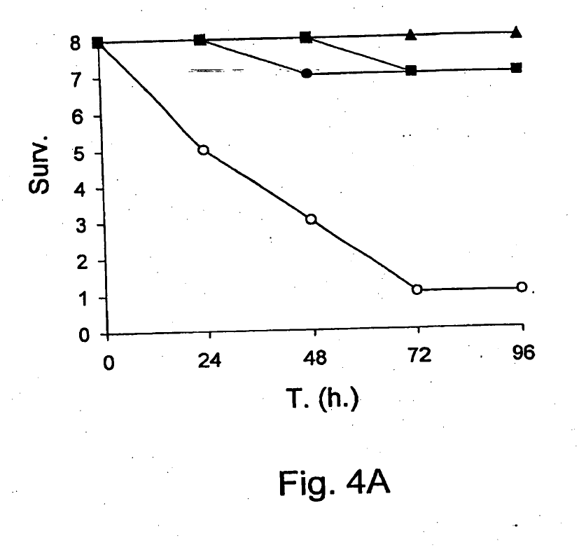

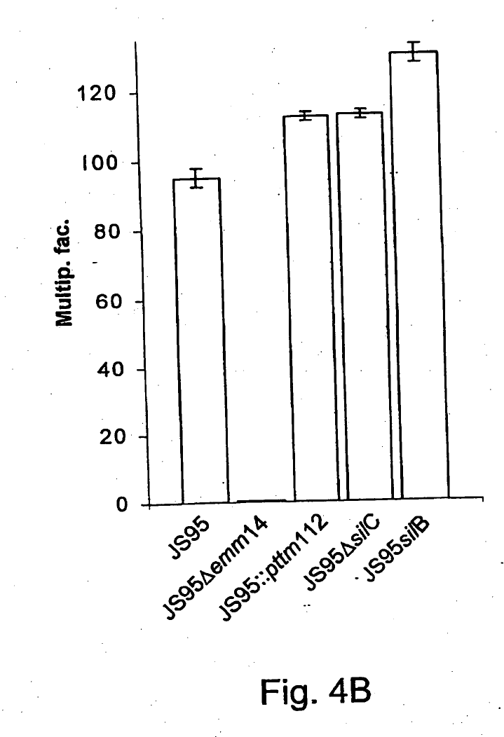

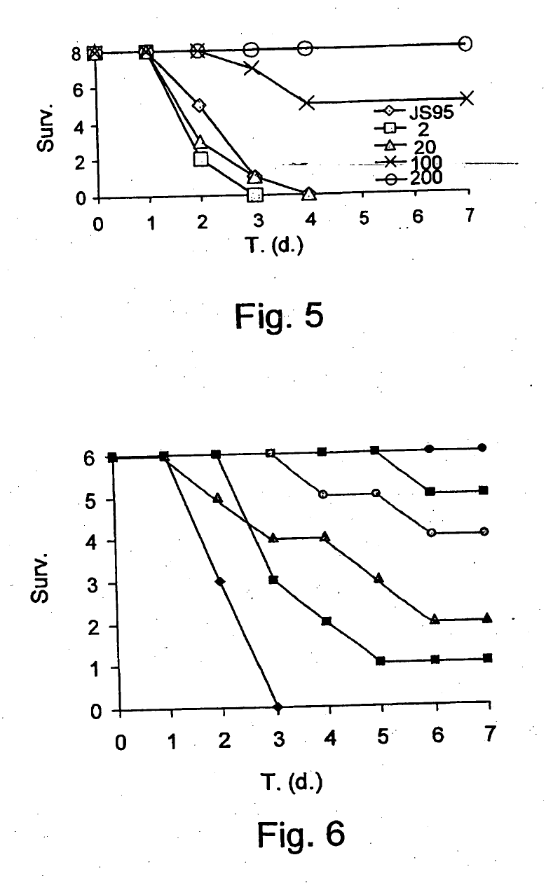

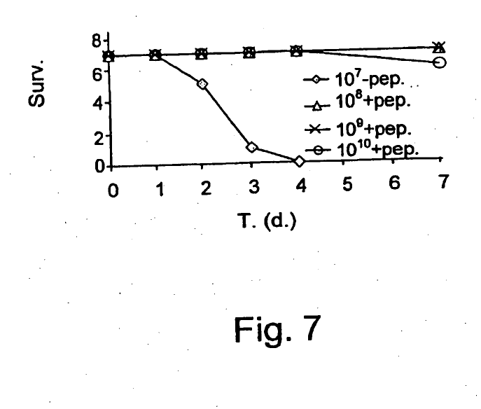

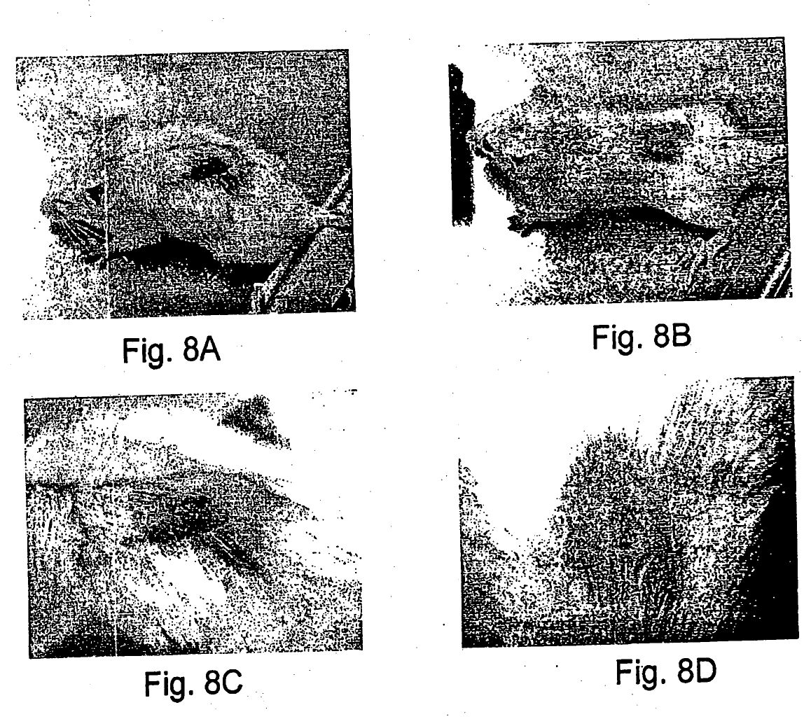

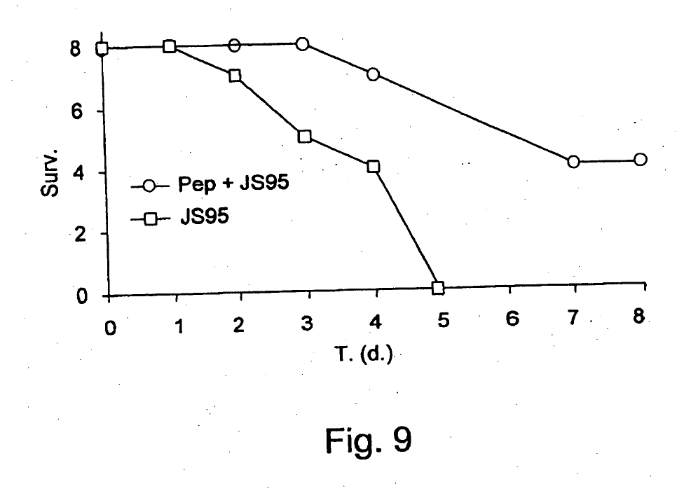

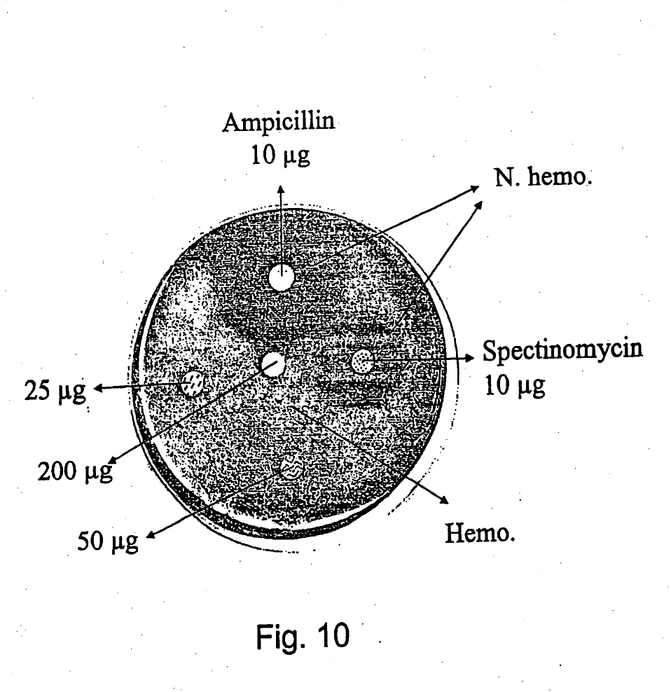

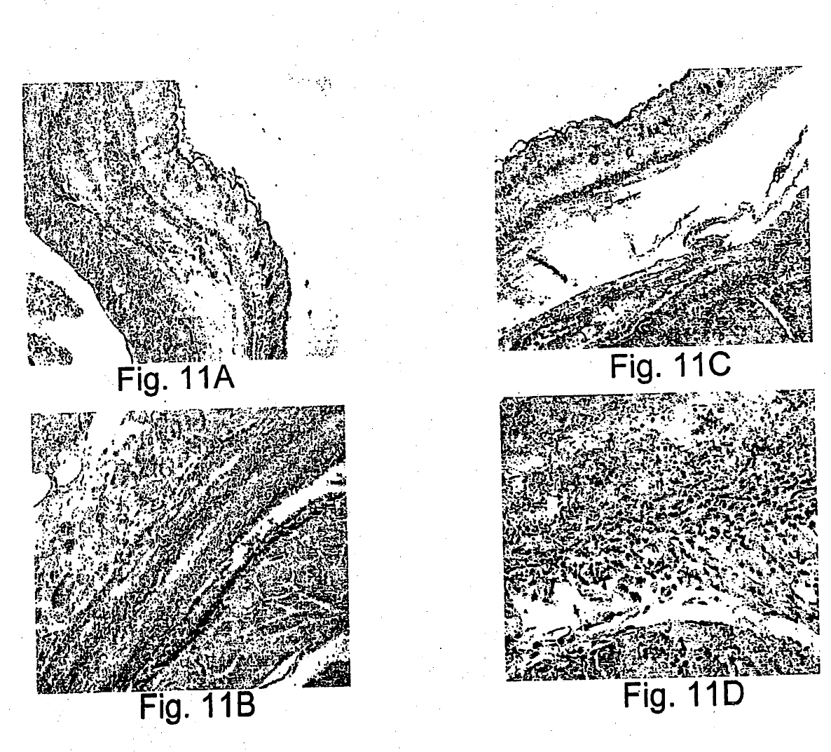

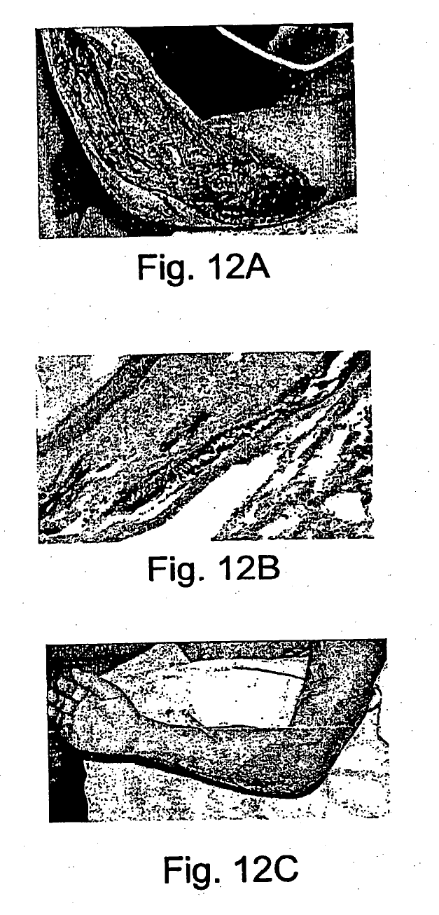

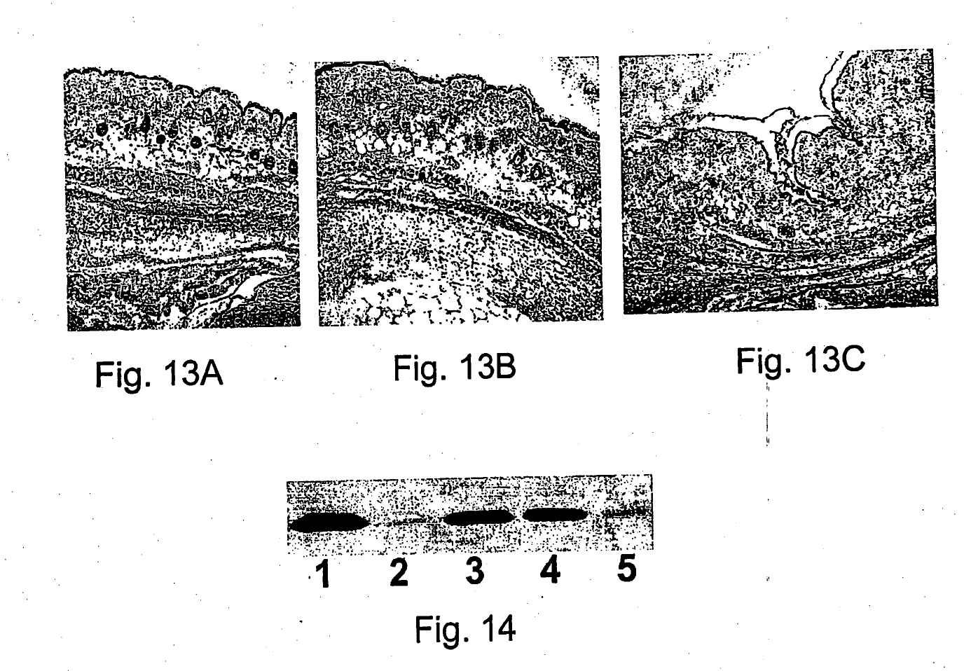

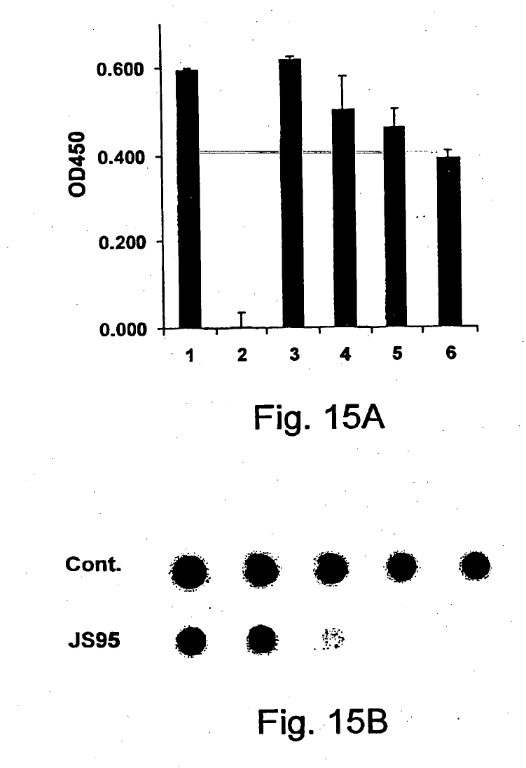

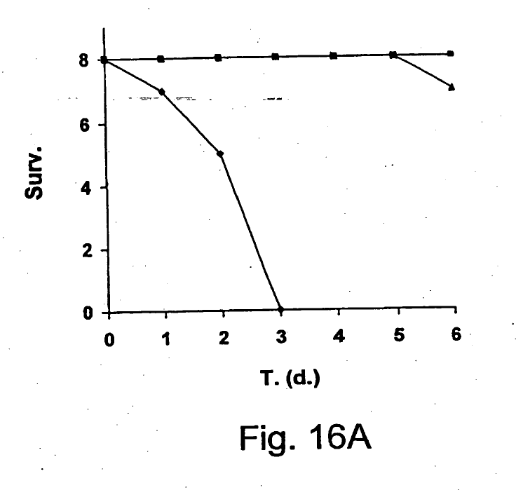

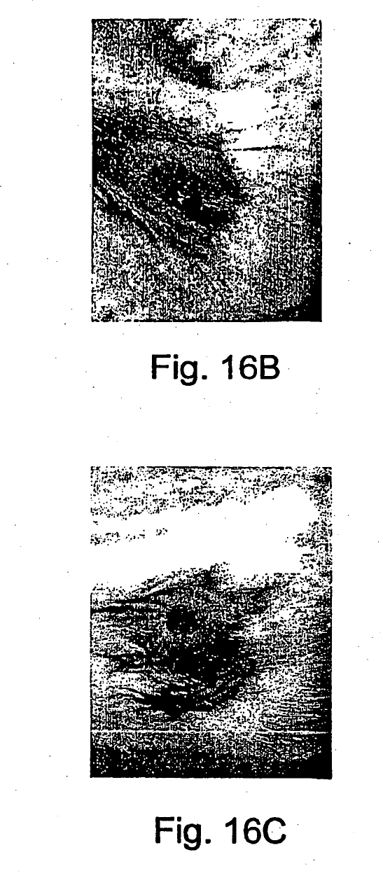

IS256 from pMGC57 was amplified using Universal and Reverse primers, and cloned into pGEM-T-easy. The resulting plasmid was digested with XbaI and SphI and the fragment containing IS256 was cloned into pUC19, generating pC256. A fragment of 677 bp of the mouse gene chop-10, which displays no homology with the M1 GAS genome, was amplified from pCHOP-10 using Universal and Reverse primers (see Table 2, SEQ ID NO. 1 and 2 respectively), and the product was cloned into pGEM-T-easy. The resulting plasmid was digested with EcoRV and XbaI and the XbaI-SmaI fragment containing a spectinomycin-resistant gene, aad9, from pFW11 was cloned. Following digestion with BglI and BglII the fragment containing aad9, the chop-10 and the E. coli origin of replication, ColE1, was cloned into the EcoRV site of pC256. The ampicillin resistance gene was removed by digestion with MunI and PvuI, generating pIS256pt. To produce a set of plasmids with different tag lengths, pIS256pt was digested with NheI (FIG. 1A) and subjected to a bi-directional deletion using Erase-a-Base system (Promega, Madison, Wis.). The resulting set of plasmids was termed pIS256pts.

B. GAS Mutagenesis

JS95 was transformed with pIS256pts (21 tags of different lengths) as described previously [Lyon, W. R. et al. EMBO J. 17:6263-6275 (1998)] and spectinomycin resistant clones were selected.

C. In-Vivo Screening of Mutants

A library of mutants each containing a tag of a different length, (input pool), was grown to an OD₆₀₀ of 0.4, adjusted to the appropriate inoculum and injected into mice [Ravins, M. et al. J. Infect. Dis. 182:1702-11 (2000)].

Output pools of mutants were isolated from the skin and from the spleen and subjected to PCR analysis using primers A and B (FIG. 1A). Primer A was fluorescently labeled with 6FAM (Biosource international, Camarillo, Calif.). The tags were resolved by electrophoresis on a sequencing gel and analyzed using ABI-Prism 3.2 GeneScan software (Perkin Elmer, Wellesley, Mass.); TAMRA 1000 (Biosource international, Camarillo, Calif.) was used as a size marker. Tag sizes of mutants present in the input and output pool were compared.

D. Identification of the Insertion Site in JS95::pttm112 and Mapping of Sil

JS95::pttm112 DNA was digested with MluI, which does not cut inside IS256pt. The digested DNA was self-ligated and transformed into E. coli. Plasmids derived from colonies resistant to spectinomycin were sequenced using primers C and D (FIG. 1A). The corresponding region in JS95 was amplified using primers SilB-f2 and SilD-r2 and sequenced using primers SilB-f1 and SilD-r1. The regions upstream and downstream of the insertion site were amplified by inverse PCR and sequenced [Ochman, H. et al. Biotechnology (N Y) 8:759-60 (1990)].

Construction of JS95 Mutants

A. JS95ΔsilC

To construct JS95ΔsilC a 1572 bp fragment containing silC, 695 bp upstream and 511 bp downstream were amplified from JS95 using the primers SilB-f2 and SilD-r2 (FIG. 3A). The PCR product was cloned into pORI280. A StyI-BspEI fragment encompassing silC was replaced by aad9. The resulting plasmid was electroporated into JS95, and mutants resistant to spectinomycin but not to erythromycin were selected. Fidelity of the replacement and the integrity of silB and silD were confirmed in one mutant, JS95ΔsilC by PCR using primers SilB-f2 and SilD-r2 (see Table 2, SEQ ID NO: 10, 11 respectively) and by sequencing.

B. JS95Δemm14

For deletion of the emm14 gene a fragment of 409 bp upstream of emm14, the ΩKm-2 and a fragment of 450 bp downstream to emm14, was cloned into pJRS233. The resulting plasmid was introduced into JS95, and erythromycin and kanamycin double resistant transformants were selected at the permissive temperature (30° C.). Growth of transformants at the nonpermissive temperature (37° C.) resulted in a single crossover insertion. A second crossover was obtained as described previously [Ashbaugh, C. D. et al. J. Clin. Invest. 102:550-60 (1998)]. Growth of transformants in the presence of kanamycin but not erythromycin indicated that a second recombination had occurred. Fidelity of the replacement was confirmed by PCR using primers allM-f and allM-r (SEQ ID NO: 28 and 29, respectively) and by Western blot using anti-M14 sera.

C. JS95silB

For insertion inactivation of silB, an intragenic fragment of silB was amplified using primers HK-f and HK-r (SEQ ID NO: 14, 15, respectively), and cloned into pJRS233. The resulting plasmid was introduced into JS95 and erythromycin-resistant transformants were isolated. The disruption of silB was verified by PCR analysis using primers SilB-f3 and SilD-r2 (SEQ ID NO: 12, 11, respectively).

D. JS95silD

For the insertion inactivation of silD, a 754 bp intragenic region of this ORF was amplified using the primers ABC1-f and ABC1-r (SEQ ID NO: 16, 17, respectively), and cloned into pFW11. The resulting plasmid was introduced into JS95 by electroporation and spectinomycin-resistant transformants were isolated. The disruption of silD was verified by PCR analysis using primers SilB-f1 and SilD-r2 (SEQ ID NO: 8, 11, respectively).

Blood Survival Assay,

The ability of GAS to survive in human blood was tested by the direct bactericidal test of Lancefield as described by us previously [Moses, A. E. et al. Infect. Immun. 65:64-71 (1997)]. In brief, 0.6 ml of freshly drawn heparinized human blood was mixed with 0.2 ml of GAS grown to OD₆₀₀ of 0.16 and diluted in THY 1: 40,000 so that the initial number of bacteria in the assay was approximately 100 to 300 CFU. Plating bacterial culture on THY plates and counting CFU determined the exact number of bacteria added to the blood. The mixture was incubated at 37° C. for 3 h with end-over-end rotation, after which, bacteria were plated on THY plates and CFU were counted. Multiplication factor was calculated by dividing the number of CFU after 3 hours with the initial CFU added to blood.

Patients:

Case 1: A 40-year-old previously healthy man was admitted one week after sustaining a minor injury to the left elbow, with fever, excruciating pain and swelling of the arm. CT of the arm showed infiltration of the subcutaneous fat around the elbow consistent with cellulitis and fasciitis. IV cefazolin was started Over the next few hours his general condition deteriorated; blood pressure dropped to 70/40. The patient was taken to the operating room where a surgical incision was performed from the mid forearm to the mid upper arm over the ulnar aspect. The fascia was found to be necrotic with a noticeable sparing of underlying muscle. A fasciotomy was performed (FIG. 12A). The wound was left open with wet saline dressings. GAS grew from blood and tissue cultures. Histopathology revealed necrosis of fascia, abundant bacteria but no neutrophil infiltration (FIG. 12B). Therapy was changed to penicillin and clindamycin. The patient was supported in the intensive care unit for 6 days and gradually recovered. Over the following ten days the patient returned to the operating room for repeated debridement of the wound. Once granulation tissue covered the wound a skin graft was applied and the arm regained complete function (FIG. 12C).

Case 2: An 84-year-old woman with diabetes mellitus on chronic steroid therapy for polymyalgia rheumatica was admitted because of fever and mental status changes for 3 days. On examination she was lethargic, tachycardic with normal blood pressure. Her right foot had chronic dry necrotic ulceration of two toes. The calf was swollen with several hemorrhagic bullae. Her leukocyte count was 2.2×10⁹/L with normal platelets. Clindamycin and ciprofloxacin were started. Blood cultures grew GAS on day 2 of hospitalisation. Over the next three days the patient's leg became progressively more swollen with signs of necrosis. On her fifth hospital day she had an above knee amputation. Histopathology disclosed widespread areas of necrotic muscle and subcutaneous tissue with many thrombosed blood vessels. In the area of muscle necrosis, an abundance of bacteria was noted without a neutrophil infiltration. After surgery the patient developed pneumonia, was in the intensive care unit and eventually died two weeks after admission.

Isolation and Identification of GAS Isolates

The Hadassah Hospital clinical microbiology laboratory identified the isolates from the surgical debrided tissues and blood as GAS by standard procedures. The M14 identification was made by serologic typing at the Israel Ministry of Health Streptococcal Reference Laboratory and verified by M-protein (emm) genotyping [Beall, B. et al. J. Clin. Microbiol. 34(4): 953-8 (1996)]. The presence of the sil locus in these strains was confirmed by PCR as previously described [Hidalgo-Grass, C. et al. Mol. Microbiol. 46(1): 87-99 (2002)], and the characteristic missense mutation changing the SilCR start codon was verified by direct DNA sequencing of the PCR amplicon. The GAS isolate from case 1 (JS95) was selected for further studies.

Cultures of GAS Strains

Typically, GAS strains were cultured in Todd-Hewitt broth supplemented with 0.2% (w/w) of yeast extract, (THY). In assays of chemokine proteolysis DMEM tissue culture medium supplemented with 10% fetal calf serum (FCS) was substituted for THY.

Serum was pretreated with PMSF followed by cysteine quenching and heat inactivation at 56° C. in order to eliminate the possibility of chemokine proteolysis by FCS itself.

SilCR Peptide Synthesis

The predicted mature form of SilCR containing the last 17 aa of the C-terminus was synthesized by the solid-phase technique [Merrifield, R. B. Science 232:341-347 (1986)] using a peptide synthesizer (Model 433A, Applied Biosystems Inc.).

Determination of the Effect of SilCR on Chemokines Proteolysis

A. Cell culture of JS95 strain was grown in THY to an OD₆₀₀ of 0.2. To determine the effect of SilCR on chemokine proteolysis, the culture was split into two equal fractions, of which one received SilCR (25 μg/ml). Both fractions were further incubated until they reached an OD₆₀₀ of 0.3. Bacteria were washed twice with sterile PBS and once with DMEM containing 10% FCS. The washed pellets were re-suspended in DMEM supplemented with FCS to their original volumes. SilCR (50 μg/ml) was added to the fraction that was grown in the presence of SilCR, and the two fractions were further incubated for 1 h at 37° C. Supernatants were collected after centrifugation. The proteolysis reaction contained: 0.08 ml of each supernatant, 100 ng of recombinant human IL-8/CXCL8 (R&D systems, Minneapolis, Minn.) or 30 ng of recombinant murine MIP-2 (R& D systems, Minneapolis, Minn.), 20 mM MOPS pH=7.2 in a final volume of 0.1 ml. The reaction was conducted for 1 h at 37° C., and stopped by boiling for 1 min.

B. For testing protease inhibitors, the supernatants were pre-incubated with the inhibitor for 30 min at 37° C., followed by the proteolysis reaction. The following concentrations of inhibitors were used: 4 mM pefabloc SC; 0.5 mM PMSF; 0.3 μM aprotinin; 1 mM benzamidine; 100 μg/ml soybean trypsin inhibitor (STI).

C. Proteolysis of chemokines was determined by ELISA and Dot-blot. IL-8 content was quantified by ELISA using the Quantikine kit for determination of human IL-8 (R&D Systems, Minneapolis, Minn.) according to the manufacturer's instructions.

Dot Blotting

Samples of 50 μl from the proteolysis reaction were diluted in PBS to a final volume of 0.5 ml and passed through a 96-wells dot-blotter onto a 0.2-μm-nitrocellulose membrane. Detection of MIP-2 was performed using anti-mouse MIP-2 polyclonal antibodies (R&D Systems Minneapolis, Minn.) followed by addition of anti-mouse IgG-HRP antibody. Dots were detected using the SuperSignal West Pico chemiluminescent detection kit (Pierce, Rockford, Ill.).

### Example 1

Lethality of NF-Causing GAS Strains in the Murine Model

The inventors conducted PPBS of invasive GAS infections in Israel during a two-year period, and identified 409 patients. In 125 cases (31%) GAS was isolated from blood. The most common illnesses were soft tissue infection (63%) and primary bacteremia (14%). Twenty-eight patients (7%) had toxic shock syndrome and thirteen (3.4%) had NF [Moses, A. E. et al. Emerg. Infect. Dis. 8:421-426 (2002)].

Examination of the NF-causing strains using the murine model indicated that all 13 strains produced an area of spreading tissue necrosis extending from the site of inoculation, as previously observed for other NF strains C. [Ashbaugh, D. et al. J. Clin. Invest. 102:560-60 (1998); Ravins, M. et al. J. Infect. Dis. 182:1702-11 (2000); Engleberg, N. C. et al. J. Infect. Dis. 183: 1043-54 (2001)]. However, only 3 strains, JS20, JS95 and JS198, were lethal when injected under the surface of the skin of mice at inocula that ranged from 4.0×10⁷ to 8.0×10⁸ colony-forming units (CFU). These 3 strains were of emm14 type as determined by emm sequencing [Facklam, R. et al. Emerg. Infect. Dis. 5:247-253 (1999)]. The JS95 strain has been used for further studies.

### Example 2

Development of the Polymorphic-Tag-Lengths-Transposon-Mutagenesis (PTTM) Method for Identification of Genes Involved in The Lethal Phenotype

To identify genes that may contribute to the lethal phenotype of JS95, the inventors have developed the PTTM system strategy (FIG. 1A). The frequency of transposition of the transposons IS256pts harboring tags of different lengths into JS95 ranged from 10³ to 10⁴ per μg DNA whereas the frequency of integration of the entire plasmids was negligible.

The PTTM strategy (FIG. 1A) is similar to that of the original STM [Hensel, M. et al. Science 269:400-3 (1995)] except for the following differences: (a) the DNA tags are derived from a single sequence but are different in size while in STM the tags are of the same size but have different sequences; (b) the comparisons of the output pools to the input pools is conducted by gel electrophoresis instead of DNA hybridization and; (c) the E. coli origin of replication in IS256pts allows easy identification of the insertion sites because it permits replication of self-ligated chromosomal fragments containing the transposon in E. coli. To achieve a satisfactory resolution, particularly between tags of close sizes, PCR products were separated on a sequencing gel. A separation of tags isolated from the skin and the spleen of a mouse challenged with a pool of 21 different mutants (harboring tags of 21 different sizes) is shown in FIG. 1B. In this experiment the inoculum of the input pool was 2×10⁶ CFU. The mouse was sacrificed 4 days after inoculation and the output pools from the skin and the spleen were compared. One of the mutants, designated JS95::pttm112, survived in the skin but failed to spread into the spleen. JS95::pttm112 was subjected to further analysis.

### Example 3

Characterization of JS95::pttm112 in the Murine Model

The ability of JS95 and that of JS95::pttm112 to cause a fatal infection were compared using a high dose of ˜5×10⁸ CFU. Mice challenged with JS95 died at a rate that was significantly higher (n=8; P=0.001, Life Table analysis, Logrank test, two groups comparison) than the rate observed for mice challenged with JS95::pttm112 (FIG. 2A).

To compare the ability of JS95 and of JS95::pttm112 to spread into internal organs two groups of mice (n=3) were inoculated with a dose of ˜2.0×10⁷ CFU. Forty-eight, 72 and 96 hours after inoculation the amount of recovered streptococcal CFU in spleens was determined. The amount of JS95 significantly increased during 48 to 96 hours after injection, reaching a value of about 4000 CFU (FIG. 2B). In contrast, the amount of recovered JS96::pttm112 remained low and only about 100 CFU were found in the spleen 96 hours after injection (FIG. 2B). JS95 and JS95::pttm112 survived in similar numbers in the skin; 1 to 3×10⁷ CFU of both strains were recovered after 96 hours. JS95::pttm112 recovered from the skin and from the spleen remained spectinomycin-resistant (not shown). These results suggest that the transposon in JS95::pttm112 was inserted into a DNA region that is involved in the ability of JS95 to invade and cause a lethal infection, thus the corresponding locus was termed sil (GenBank accession number AF493605) for streptococcal invasion locus.

### Example 4

Genomic Organization of the Sil Locus

The inventors have found that sil is composed of five ORFs, which were termed silA-E. Analysis of the sequences flanking the insertion site in JS95::pttm112 and sequencing of the corresponding region in JS95 revealed that the transposon was inserted into silC (FIG. 3A). silC consists of 120 nucleotides and does not exhibit a significant homology with any known sequence. It is preceded by a combox promoter [Morrison, D. A. et al. Res. Microbiol. 151:445-51 (2000)], TACGAATA, with a single transversion of G →T, and a T-rich region at −25 (FIG. 3A). Genes driven by the combox promoter are linked to the quorum-sensing regulated competence in streptococci, usually are late transcribed and form the DNA uptake mechanism [Lacks, S. A and Greenberg, B. Mol. Microbiol. 42:1035-1045 (2001); Morrison, D. A. et al. Res. Microbiol. 151:445-51 (2000)]. Recently, the genome of the M18 strain MGAS8232 was sequenced and a highly homologues region to sil locus was identified [Smoot (2002) ibid.]. In this strain, silCR, which overlaps silC (FIG. 3B), was suggested to be transcribed from the reverse strand. SilCR encodes a 41 amino acid peptide with a leader containing a double-glycine and the motif RKK at the C-terminal [Smoot (2002) ibid.]. These properties typify signaling peptides in quorum-sensing competence regulons of streptococci [Miller, M. B. Annu. Rev. Microbiol. 55:165-199 (2001)]. In JS95 this sequence is identical, except that the ATG initiation codon is replaced by ATA (FIG. 3B), as was confirmed by multiple sequencing. The alignment of silC with silCR is shown in FIG. 3B. These two small ORFs share 91 nucleotides.

Genes homologues to sil were not detected in the M1 chromosome [Ferretti, J. J. et al. Proc. Natl. Acad. Sci. U.S.A. 98:4658-63 (2001)], yet sil exhibited high similarity with genes of S. pneumoniae. Analysis of silA and silB showed that they encode a putative two-component system (TCS) consisting of a response regulator and a histidine kinase (HK) sensor, respectively. SilA is 51% identical and 71% similar to S. pneumoniae response regulator BlpR, whereas SilB is 32% identical and 52% similar to the HK sensor, BlpH; BlpR and BlpH are a TCS of a locus that is predicted to produce a bacteriocin [De Saizieu (2000) ibid.]. A promoter, located upstream of silA, probably drives the transcription of silA and silB (FIG. 3A). silD and silE encode putative ABC transporters that are highly homologous to BlpB/ComB, and to ComA [Hui, F. M. et al. Gene 153:25-31 (1995); De Saizieu (2000) ibid.]. SilD exhibits 56% identity and 76% similarity with BlpB, and 33% identity and 55% similarity with ComB. A homologue of silD in the strain MGAS8282 is truncated and composed of two ORFs, SpyM18_{—}0541 and SpyM18_{—}0542 [Smoot (2002) ibid]. SilE shares 67% identity and 83% similarity with ComA.

Downstream of the sil locus, the inventors have identified an ORF homologous to S. pneumoniae blpM comprising a gene that encodes a predicted bacteriocin peptide [De Saizieu (2000) ibid.] that was designated blpM^{H} (FIG. 5C). By sequence alignment, a truncated blpM homologue (blpM^{T}) located upstream of SPy0484 (FIG. 3C) was identified in the M1 chromosome. To characterize the region further downstream of sil, primers hybridizing to blpM^{H} in JS95 and to SPy0488 and mutR in M1 (FIG. 3C) were designed. The amplified fragments in JS95 (blpM^{H}-SPy0488 and blpM^{H}-mutR) were identical in size to those expected for the M1 chromosome, indicating that the region downstream of the sil locus in JS95 is homologues to section 36 of the M1. The IS1562 [Berge (1998) ibid.] was found upstream of silA. To identify the region further upstream to IS1562 the inventors have designed primers annealing to IS1562 in JS95, and to era and SPy0479 in the M1 chromosome, respectively (FIG. 3C). The PCR analysis showed that in JS95 sil locus is also flanked upstream by sequences present in section 36 of M1 chromosome, but JS95 contains additional regions of 2 kb located between IS1562 and SPy0479 and 4 kb between era and SPy0479.

### Example 5

The Role of Sil in Virulence

Involvement of SilC in Virulence

To establish that silC is involved in virulence and to rule out a possible polar effect caused by the transposon insertion in JS95::pttm112, the mutant JS95ΔsilC, in which silC was deleted and replaced with a spectinomycin resistance cassette aad9 was next constructed. In addition, silB was inactivated by insertion mutagenesis, yielding JS95silB. Subsequently, the different mutants were tested for their ability to kill mice using a challenge of ˜5×10⁷ CFU (FIG. 4A). JS95ΔsilC, JS95silB and JS95::pttm112 were severely attenuated in the ability to cause a lethal infection compared to JS95 (n=8; P<0.0001, Life Table analysis, Logrank test, four groups comparison). These results indicate that the sil locus significantly contributes to an invasive and fatal infection in the animal model.

Survival in blood by preventing phagocytosis is a critical trait in virulence of GAS [Lancefield, R. C. J. Exp. Med. 107:525-544 (1957)]. To assess the contribution of sil to blood survival, the JS95 strain and its derived mutants were subjected to the bactericidal test of Lancefield [Lancefield (1957) ibid.]. As a negative control, an M14-deficient mutant, JS95Δemm14 was constructed. As demonstrated for an M18-deficient mutant [Moses, A. E. et al. Infect. Immun. 65:64-71 (1997)], JS95Δemm14 did not survive in blood. In contrast, JS95::pttm112, JS95ΔsilC and JS95silB survived and grew to the same extent as JS95 (FIG. 4B). These results show that sil locus does not affect JS95 survival in blood.

In-Vivo Complementation of Virulence by Injecting a Mixture of Isogenic JS95 Mutants

Since sil is highly homologous to the quorum-sensing regulons blp and com [Hui, F. M. et al. Gene 153:25-31 (1995); De Saizieu (2000) ibid.], the inventors have next examined the possibility to complement the virulence of JS95ΔsilC in-vivo by injecting it in combination with the isogenic M-deficient mutant, JS95Δemm14. Consistent with the report of Ashbaugh et al. [Ashbaugh, C. D. et al. J. Clin. Invest. 102:550-60 (1998)], JS95Δemm14 was a virulent. Mice injected with JS95Δemm14 developed a local lesion at the site of inoculation, but only one mouse out of 24 challenged died (Table 3, Group 1). In contrast, JS95 was highly virulent in mice Cable 3 Group 5). As expected, JS95ΔsilC was attenuated in its ability to kill mice, two out of 24 mice challenged died within 72 hours and another 2 between 72 to 96 hours (Table 3, Group 2). However, when mice were injected at one site with a premixed inoculum of JS95ΔsilC and JS95Δemm14, a lethal infection developed rapidly. Out of 32 challenged mice, 23 died within 72 hours and another 6 between 72 and 96 hours (Table 3, Group 3). In contrast, when mice were challenged with the same mutants, but each mutant was injected separately into opposite flanks, lethality was only slightly higher than in mice challenged with JS95ΔsilC alone. Two out of 16 mice challenged died after 72 hours and another 3 died between 72 and 96 hours (Table 3, Group 4). A mixed model analysis of variance was performed in which “Group” was a fixed effect, “Experiment” was a random effect, and the response variable was percent survival at 72 hours and 96 hours, respectively. “Group” had a significant effect on mortality at both of these times (P=0.0012, 72 hours; P<0.0001, 96 hours). A pair-wise comparison between each of the five “Groups” was performed, using the Bonferonni correction for multiple comparisons. The difference in the rate of mortality of Groups 3 and 5 compared to Groups 1, 2 and 4 was statistically significant at both 72 and 96 hours.

The number of GAS CFU in the spleen of mice in Groups 3 and 4 was next examined. Seventy-two hours after inoculation the amount of CFU in Group 3 (n=3) was 25,305±2269, whereas in Group 4 (n=3) only 1720±120. The complementation of the full virulent phenotype observed only for the premixed inocula, suggests that signaling occurs between JS95ΔsilC and JS95Δemm14.

TABLE 3
Complementation of virulence.

| | | | Animals | | Inoculum | Mortality (%)^{a} | |
|---|---|---|---|---|---|---|---|
| Group | Strain | Injection | (n) | Experiments | (10⁸ CFU) | 72 hou | 96 hours |
| 1 | JS95Δemm14 | One site | 24 | 3 | 1.6-29.0 | 4.1 | 4.1 |
| 2 | JS95ΔsilC | One site | 24 | 3 | 1.0-15 | 8.3 | 16.6 |
| 3 | JS95Δemm14 and JS95ΔsilC (premixed) | One site | 32 | 4 | 1.2-2.2 | 71.8 | 90.6 |
| 4 | JS95Δemm14 and JS95ΔsilC | Two sites^{b} | 16 | 2 | 1.2-4.1 | 12.5 | 31.2 |
| 5 | JS95 | One site | 21 | 3 | 1.0-2.3 | 76.2 | 90.5 |

^{a}The difference in the rate of mortality of groups 3 and 5 compared to groups 1, 2 and 4 was statistically significant at both 72 and 96 hours.
^{b}Injected into opposite flanks

### Example 6

A 17 Amino Acid Peptide Transcribed from silCR Exhibits Anti-Virulent properties

As indicated herein above, the sil locus contains at least five genes (silA-E) and is highly homologous to the quorum-sensing competence regulons of Streptococcus pneumoniae. silA and silB encode a TCS and silD and silE two ABC transporters: silC was found to be a small ORF of an unknown function. Insertion inactivation of silB and deletion of silC resulted in mutants of a considerably reduced ability to spread from the site of inoculation into internal organs and thus to cause lethal infection.

In order to find out whether sil locus displays homology with other GAS sequences, the sil sequence was aligned with the genome sequence of an M1 strain of Streptococcus pyogenes [Ferretti et al. (2001) ibid.]. The results indicated that it does not contain any locus homologous to sil.

As indicated herein before, recently, Smoot et al. [Smoot (2002) ibid] have reported the genome sequence of serotype M18 group A Streptococcus (GAS) strains. As described in Example 4, a detailed alignment of the M18 sequence with the sil A-E locus performed by the inventors revealed that the M18 strain contains a locus highly homologous to sil. Surprisingly, a putative open reading frame highly overlapping silC is probably transcribed in the M18 strain from the reverse strand. This ORF encodes a predicted 41 amino acid precursor peptide that has a GlyGly cleavage site common to peptides processed and transported by the ABC transporter system. The 41 amino acid peptide may be cleaved to form a predicted 17 amino acid mature peptide with properties that typify signaling peptides in quorum-sensing competence regulons of streptococci [Smoot (2002) ibid.]. In the sequence of the highly invasive strains isolated by the inventors, the ATG initiation codon was found to be replaced by ATA. Replacement of the ATG codon by an ATA might prevent translation of the predicted 41 amino acid precursor peptide, and therefore the formation of the 17 amino acid peptide. The unexpected elimination of the predicted 17 amino acid (aa.) peptide formation in the highly virulent strains of the invention, together with the involvement of this locus in virulence, as found by the invention, have led the inventors to hypothesize that the peptide may be involved in attenuation of virulence.

Therefore, the inventors next tested the peptide effect on virulence of the highly invasive strain of the invention. A 17 amino acid peptide, corresponding to the amino acid sequence encoded by the SilCR ORF, was synthesized and purified. Mice were challenged with the highly virulent strain (JS95) in the presence and absence of the 17 amino acid synthetic peptide DIFKLVIDHISMKARKK (also denoted by SEQ ID NO: 32), of a 70% purity. As shown by FIG. 5, the peptide protected mice in a dose-dependent fashion. A partial protection was obtained when 100 μg of the peptide were injected together with the challenging bacteria (10⁷ CFU), and complete protection was achieved by injection of 200 μg of the peptide.

Similar results were observed when a peptide of 90% purity was used As shown by FIG. 6, the protective effect of SilCR was dose-dependent; evident at a peptide dose as low as 3 μg, and reached a plateau level of protection at 50 μg.

The ability of the peptide to protect mice inoculated with increasing doses of the bacteria ranging from 10⁷ to 10¹⁰ CFU was next examined. The results showed an effective protection of mice challenged with bacteria in all tested doses (FIG. 7). Mice challenged with a low dose of 10⁷ CFU, in the absence of the peptide, died within 4 days after challenge. The peptide completely protected mice challenged with 10⁷ and up to 10⁹ CFU, and only one mouse died when mice were challenged with 10¹⁰ CFU (FIG. 7). Results were consistent even after 21 days of follow up (data not shown).

FIG. 8 shows a further example of the protective ability of the peptide of the invention. Mice were injected under the surface of the skin with 2×10⁸ of M14 GAS strain, JS95, without (FIG. 8A) or with 25 μg of the highly purified (90% purity) 17 amino acid peptide (SEQ ID NO: 32) (FIG. 8B). An extensive necrotic area of the skin surrounded by edema was observed in mice injected with bacteria in the absence of the peptide (FIG. 8A, 8C), all mice of this group died 24-48 hours post infection. In contrast, mice infected with the bacteria treated with 25 μg of the 17 amino acid peptide, had a small abscess surrounded by a normal skin (FIG. 8B, 8D), all mice of this group survived.

In order to examine the ability of the 17 amino acid peptide (SEQ ID NO: 32) to protect against bacterial challenge, mice were injected with 200 μg of this peptide 24 hours prior to bacterial challenge. As shown by FIG. 9, the 17 amino acid peptide protects 50% of the mice when injected 24 hours before bacterial challenge of 4×10⁸ CFU of JS95 strain.

Taken together, these results clearly show that the peptide of the invention man protect mice even against extremely high doses of a highly invasive GAS strain and over a long time period.

The inventors next examined whether the 17 amino acid peptide of the invention protects mice challenged with highly virulent strains by inhibition of bacterial growth. Different concentrations (1000-1500 CFU) of GAS strain JS95 were spread on the surface of a blood-agar plate and incubated at room temperature. Disks loaded with different concentrations of antibiotics (control) or peptide (25, 50 and 200 μg) were placed on the surface. As shown by FIG. 10, bacterial growth was observed even in the presence of highest concentrations of the peptide (200 μg). These results indicate that the peptide does not mediate inhibition of bacterial growth, and therefore may be involved in signaling pathways mediating virulence. Since the peptide exhibits properties that are common for peptides quorum sensing peptides regulating competence in streptococci Pozzi, et al. [J. Bacteriol. 178:6087-6090 (1996); Havarstein, et al. J. Bacteriol. 179:6586-6584 (1997)] it is plausible that the peptide acts on a TCS. This in turn activates a signal transduction process in the bacteria leading to attenuation of virulence.

### Example 7

A 17 Amino Acid Peptide Transcribed from silCR Allows Recruitment Of PMN to the Site of Infection

As shown by FIG. 11, the inventors have further found that neutrophils (PMN) are not present at the site of the skin lesion caused by inoculation of virulent strain of GAS JS95. Twenty-four hours after inoculation a destruction of dermis and epidermis was observed. Bacteria accumulated under the skin and caused necrosis of the fascia (FIG. 11A). Bacteria occupied the region corresponding to the fascia and no PMN was observed in this region (FIG. 11B). In contrast, staining performed in skin samples obtained from mice challenged with the same bacteria in the presence of the peptide revealed a significant recruitment of PMN to the area of the infection. Without being bound by theory, this finding led the inventors to hypothesize that recruitment of PMN to the site of infection and activation of the immune response allows the mice to survive the infection.

While injection of the SilCR peptide alone did not induce neutrophil influx, the inventors have observed abundant neutrophil infiltration into the necrotic fascia and the underlying tissues in mice challenged with JS95 and SilCR (FIG. 11D). This is in a sharp contrast to the paucity of neutrophil influx in mice challenged with JS95 alone (FIG. 11B). Thus, a rapid recruitment of neutrophils appears necessary to confine GAS infection, subsequently preventing a systemic GAS dissemination.

### Example 8

GAS Infection of Human Cases and Mouse Model are Characterized By the Paucity of Neutrophil Influx into the Necrotic Site of Infection

Tissues obtained from two human subjects suffering from NF infections (cases 1 and 2), by surgical debridement. The samples were examined histologically. As shown by FIG. 12B, these samples were characterized by the presence of massive amounts of bacteria and lack of infiltrating neutrophils. In contrast, the viable tissue surrounding the site of necrosis revealed the presence of neutrophils but no bacteria. To gain further insight into the lack of neutrophil recruitment the inventors next used the mouse model of human soft-tissue infection [Ashbaugh, C. D. et al. J. Clin. Invest. 102(3):550-60 (1998)]. AB shown herein before by FIG. 8A, mice injected with 10⁸ CFU of M14 GAS strain JS95 became severely sick within 12 h (not shown), and after 24 h appeared lethargic, with mottled hair (FIG. 8A) and closed eyes. Within 24 h, mice developed an area of spreading tissue necrosis extending from the site of inoculation to the surrounding skin and into the deep subcutaneous tissues (FIG. 11B). The mice invariably succumbed to the infection after 48 to 96 h. To characterize the pathological evolution of the infection process, two mice were sacrificed 3, 6, 12 and 24 h after inoculation with strain JS95. As shown by FIG. 13A, three hours after injection, bacteria appeared in the fascia, which became necrotic, without concomitant necrosis of the skin or subcutaneous tissues. Only mild infiltration of neutrophils was detected at the injection site, similar to control injections with PBS alone. Six hours after challenge (FIG. 13B), necrosis extended from the fascia to the hair follicles. Twelve hours after challenge, extensive necrosis of fascia, dermis and epidermis was observed, along with massive numbers of bacteria (FIG. 13C). A paucity of neutrophil infiltration to the fascia and surrounding tissues was noticed 12 h after inoculation while neutrophils were completely absent at 24 h after inoculation (FIG. 11B). These histopathological findings in the human cases and in the murine model suggested that the high invasiveness of the M14 GAS strains might in part reflect an impaired host neutrophil response that fails to contain the localized bacterial infection.

### Example 9

GAS Induces Chemokine Degradation by a Serine-Class Protease

IL-8 is a potent C-X-C family chemokine and the best-studied neutrophil chemoattractant in humans [Luster, A. D. C. N. Enl. J. Med. 338(7):436-45 (1998)]. The inventors therefore next tested whether a GAS-encoded proteolytic activity may cause IL-8 degradation. Supernatant of JS95 grown to early log phase was incubated with IL-8 in the absence and presence of class-specific protease inhibitors. As shown by the Western blot analysis of FIG. 14, while JS95 supernatant effectively degraded IL-8, the irreversible serine protease inhibitor pefabloc SC almost completely abolished IL-8 degradation. Further analysis has shown that aprotinin, which effectively inhibits trypsin, chymotrypsin, plasmin and kallikrein [Katunuma, N. et al. in: Biological functions of proteases and inhibitors. Editor(s): Fritz, H. (Munich); Katunuma, N. (Tokushima); Suzuki, K. (Tokyo); Travis, J. (Athens, Ga.) Tokyo: Japan Scientific Societies Press, Pages: XII +274 (1994)], completely inhibited IL-8 degradation, supporting the notion that the protease responsible for IL-8 degradation is of the serine-class (FIG. 15A). Finally, benzamidine and soybean trypsin inhibitor also completely abrogated IL-8 degradation (FIG. 15A), supporting the serine-class assignment, and further suggesting that IL-8 is degraded by a trypsin-like GAS protease. Inhibitors of the cysteine-class metallo-class of leucine aminopetidase and of aminopeptidase P, did not inhibit IL-8 degradation (not shown).

In the mouse, macrophage inflammatory protein (MIP)-2 is a functional homologue of human IL-8²¹ [Van Damme, J. et al. J. Leukoc. Biol. 62(5):563-9 (1997)], thus, the inventors have examined the ability of GAS supernatant to degrade MIP-2. The supernatant of JS95 degraded more than 80% of the recombinant MIP-2 protein (FIG. 15B). The MIP-2 degradation was blocked by pretreatment of the supernatant with pefabloc SC, aprotinin, benzamidine, and STI, suggesting that, like IL-8 MIP-2 is degraded by a trypsin-like GAS protease.

### Example 10

SilCR Peptide Induces Abundant Neutrophils Infiltration into the Necrotic Facia of Infected Subjects

The inventors have further discovered that when JS95 is grown in the presence of SilCR, the strain exhibited markedly reduced IL proteolytic activity (FIG. 15A). This result reflects an action of SilCR upon the bacterium, as the purified SilCR peptide did not itself block IL-8 proteolysis (FIG. 14). It should be further noted that the inventors have found that SilCR does not inhibit GAS growth as assessed in THY medium and on blood agar plates (FIG. 10).

The inventors have next sought to explore the effect of SilCR, which inhibits GAS-mediated chemokine degradation, on the progression of GAS infection in the murine model.

Therefore, mice were challenged with JS95 in the absence and presence of SilCR. As shown by FIGS. 8B and 8D, dramatic therapeutic effect was achieved when mice were challenged with JS95 and SilCR. Mice appeared sick in the first 12 h, however after 24 h they moved vigorously and their hair was less mottled than in mice challenged with JS95. As shown by FIG. 8B, the lesions of mice challenged with JS95 and SilCR were significantly smaller, less necrotic and superficial, with defined borders (FIG. 8B compared to FIG. 8A). As shown by FIG. 16A, even minimal amount of 3 μg of the 90% purified peptide have protected mice even against a JS95 challenge as high as 10¹⁰ CFU.

GAS of M1 and M3 serotypes are commonly associated with invasive disease and NF, and each lack the SilCR gene in their locus. GAS M1 strain 340, isolated from a patient with TSS, produced necrotic lesions in the inventors NF model (FIG. 16B). Co-injection of SilCR with of the M1 340 strain resulted in lesions of a significantly reduced size and duration (FIG. 16C). Similarly, reduced lesions were produced when SilCR was injected together with an M3 strain (data not shown).

1. (canceled)

2. (canceled)

3. (canceled)

4. (canceled)

5. (canceled)

6. (canceled)

7. (canceled)

8. (canceled)

9. (canceled)

10. A method of inhibiting an invasive and/or non-invasive infection of Gram-positive pathogenic bacteria in a mammalian subject, comprising administering to said subject an inhibitory effective amount of an isolated and purified peptide or of a composition comprising the same, which peptide comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605 or any functional fragment, analog or derivative thereof.

11. A method of preventing and/or treating a Gram-positive bacteria invasive infection related pathologic disorder comprising administering to a mammalian subject in need of such treatment a therapeutically effective amount of an isolated and purified peptide or of a composition comprising the same, which peptide comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605 or any functional fragment, analog or derivative thereof.

12. The method according to claim 10, wherein said peptide comprises an amino acid sequence substantially as denoted by SEQ ID NO: 32 or any functional fragment, analog or derivative thereof.

13. The method according to claim 12, wherein said Gram-positive bacteria is Streptococcus sp. selected from the group consisting of Group A Streptococcus (GAS) bacteria, Group B Streptococcus bacteria and Group B Streptococcus bacteria.

14. The method according to claim 13, wherein said peptide inhibits the spreading of GAS bacteria and/or tissue necrosis and/or lethal effect caused by said bacteria.

15. The method according to claim 10, wherein said invasive infection leads to one of soft tissue infection, bacteremia, septicemia, toxic shock syndrome (TSS) and necrotizing fasciitis (NF) and the non-invasive infection leads to one of rheumatic fever and acute glomerulonephritis.

16. The method according to claim 14, wherein said GAS bacteria is one of Streptococcus pyogenes, Streptococcus pneumoniae, Staphylococcus aureus and oral Streptococci.

17. The method according to claim 16, wherein said GAS bacteria is a virulent strain of Streptococcus pyogenes.

18. The method according to claim 17, wherein said peptide is in the form of a dimer, a multimer or in a constrained conformation.

19. The method according to claim 18, wherein said peptide is conformationally constrained by internal bridges, short-range cyclization, extension or other chemical modification.

20. The method according to claim 11, wherein said GAS invasive infection related pathologic disorder is one of soft tissue infection, bacteremia, septicemia, toxic shock syndrome (TSS) and necrotizing fasciitis (NF), and the non-invasive infection leads to any one of rheumatic fever and acute glomerulonephritis.

21. The method according to claim 10, wherein said peptide or composition comprising the same is administered to said subject prior to potential exposure to said pathogenic bacteria.

22. The method according to claim 10, wherein said peptide or composition comprising the same is administered to said subject in a single dose and or multiple doses.

23. The method according to claim 22, wherein said peptide or composition comprising the same is administered to said subject by a single route or by at least two different routes of administration.

24. The method according to claim 23, wherein said peptide or composition comprising the same is administered to said subject by at least one route selected from oral, intravenous, parenteral, transdermal, subcutaneous, intravaginal, intranasal, mucosal, sublingual, topical and rectal administration and any combinations thereof.

25. The method according to claim 24, wherein said peptide or composition comprising the same is administered to said subject subcutaneously.

26. The method according to claim 10, wherein an effective amount of said peptide is between 0.5 μg/kg to 100 mg/kg of body weight.

27. The method according to claim 26, wherein an effective amount of said peptide is between 10 μg/kg to 10 mg/kg of body weight.

28. The method according to claim 27, wherein an effective amount of said peptide is between 300 μg/kg to 5 mg/kg.

29. (canceled)

30. A method of disinfecting an environment and/or preventing infection caused by Gram-positive bacteria, comprising the step of applying a sufficient amount of an isolated and purified peptide or of a composition comprising the same, onto a surface of any one of medical equipment, medical devices and disposables, which peptide comprises an amino acid sequence substantially homologous to the amino acid sequence encoded by the SilCR ORF of the sil locus at position 2985 to 2863 of the complementary strand of the genomic sequence of Group A Streptococcus (GAS) bacteria specified in GenBank accession number AF493605 or any functional fragment, analog or derivative thereof.

31-62. (canceled)


SEQUENCE LISTING (32 sequences)

SEQ ID NO: 1 - DNA, 19 bp, Artificial Sequence
  m13/puc sequence primer (-20)
gtaaaaaacgacggccagt

SEQ ID NO: 2 - DNA, 16 bp, Artificial Sequence
  m13/puc reverse sequencing primer (-21) forward primer for tag amplification
aacagctatgaccatg

SEQ ID NO: 3 - DNA, 20 bp, Artificial Sequence
  Reverse primer for tag amplification
agcagttcgtagttatcttg

SEQ ID NO: 4 - DNA, 19 bp, Artificial Sequence
  Inverse PCR primer from IRr
ttatcagcaataaaccagc

SEQ ID NO: 5 - DNA, 18 bp, Artificial Sequence
  Inverse primer from IR1
aaagtcctcctgggtatg

SEQ ID NO: 6 - DNA, 20 bp, Artificial Sequence
  Inverse PCR primer from 3′ of silE
tttggcagctttgacgatgc

SEQ ID NO: 7 - DNA, 20 bp, Artificial Sequence
  Inverse PCR primer from 5′ of SilA
tcttcaagcagctgattggg

SEQ ID NO: 8 - DNA, 23 bp, Artificial Sequence
  2598-2620 in sil
ggagttggtttatcaaatgtcag

SEQ ID NO: 9 - DNA, 23 bp, Artificial Sequence
  3213-3235 in sil
atctgccacaaagactgatcaag

SEQ ID NO: 10 - DNA, 21 bp, Artificial Sequence
  2013-2033 in sil
ttattggatcggaacttacgc

SEQ ID NO: 11 - DNA, 21 bp, Artificial Sequence
  3554-3574 in sil
tgcttcccaacaacttaccac

SEQ ID NO: 12 - DNA, 22 bp, Artificial Sequence
  2088-2109 in sil
gctcgctatagtaagcaaatcg

SEQ ID NO: 13 - DNA, 18 bp, Artificial Sequence
  5871-5888 in sil
cagcgattaagcattgac

SEQ ID NO: 14 - DNA, 20 bp, Artificial Sequence
  1616-1634 in sil
acgaaaggtcaatggttcac

SEQ ID NO: 15 - DNA, 20 bp, Artificial Sequence
  2338-2357 in sil
aggtatggataagcgttgag

SEQ ID NO: 16 - DNA, 20 bp, Artificial Sequence
  3873-3894 in sil
atgacacttgttacacgtcc

SEQ ID NO: 17 - DNA, 22 bp, Artificial Sequence
  3873-3984
actagtcagcttgacgaacttc

SEQ ID NO: 18 - DNA, 19 bp, Artificial Sequence
  emm typing forward primer
tattcgcttagaaaattaa

SEQ ID NO: 19 - DNA, 20 bp, Artificial Sequence
  emm typing reverse primer
gcaagttcttcagcttgttt

SEQ ID NO: 20 - DNA, 28 bp, Artificial Sequence
  aad9 forward primer
ccatggtcctcgagctctagatcttaag

SEQ ID NO: 21 - DNA, 25 bp, Artificial Sequence
  aad9 reverse primer
ctgcaggcgcttaccaattagaatg

SEQ ID NO: 22 - DNA, 24 bp, Artificial Sequence
  6873-6896 in JS95 sil, 5096-5119 in M1
tcgatatggagataaagaaactgg

SEQ ID NO: 23 - DNA, 22 bp, Artificial Sequence
  6804-6825 in M1 section 36
aacagtgctttcaggaactcct

SEQ ID NO: 24 - DNA, 22 bp, Artificial Sequence
  10031-10052 in M1 section 36
ctaggtgcaattgaggagtcaa

SEQ ID NO: 25 - DNA, 20 bp, Artificial Sequence
  20-43 in JS95 sil, 7287-7306 section 152 in M1
tcctcgcactgttccaatag

SEQ ID NO: 26 - DNA, 20 bp, Artificial Sequence
  3580-3599 in M1 section 36
aggtggtgttggagcaggta

SEQ ID NO: 27 - DNA, 21 bp, Artificial Sequence
  1545-1565 in M1 section 36
aagaagtggtcccaatttctg

SEQ ID NO: 28 - DNA, 30 bp, Artificial Sequence
  Forward all M primer with BamHI site
cctgaaaatgaggatccttcctaaaaaacg

SEQ ID NO: 29 - DNA, 32 bp, Artificial Sequence
  Reverse all M p[rimer with PstI site
gggggctgcagagcttagttttcttctttgcg

SEQ ID NO: 30 - DNA, 20 bp, Artificial Sequence
  3′ of mga
gattccagaagcgattattg

SEQ ID NO: 31 - DNA, 21 bp, Artificial Sequence
  Leader peptide region of scpA
aatggcaagtttatcaaatgg

SEQ ID NO: 32 - protein, 17 aa, Artificial Sequence
  SilCR peptide
DIFKLVIDHISMKARKK